(19) United States
(12) Patent Application Publication
(10) Pub. No.: US 2008/0154005 A1
(43) Pub. Date: Jun. 26, 2008

(54) Hole-Injecting Material, Material for Light-Emitting Element, Light-Emitting Element, Organic Compound, Monomer, and Monomer Mixture

(75) Inventors: Tsunenori Suzuki, Kanagawa (JP); Ryoji Nomura, Kanagawa (JP); Sachiko Kawakami, Kanagawa (JP)

Correspondence Address: ERIC ROBINSON, PMB 955, 21010 SOUTHBANK ST., POTOMAC FALLS, VA, 20165, US

(73) Assignee: Semiconductor Energy Laboratory Co., Ltd., Atsugi-shi (JP)

(21) Appl. No.: 11/795,097

(22) PCT Filed: Jan. 26, 2006
(86) PCT No.: PCT/JP2006/301709

§ 371 Date: Jul. 11, 2007

(30) Foreign Application Priority Data
Jan. 31, 2005 (JP) 2005-024542

(51) Int. Cl. C08F 12/32; C07C 211/54

(52) U.S. Cl. 526/310; 564/434

(57) ABSTRACT

An object of the present invention to provide a material for a light-emitting element or a hole injecting high molecular weight material which has a sufficient hole injecting property, without using a dopant having an electron accepting property. One of materials of the present invention which can achieve the object, has a repeating unit represented by the following formula (1), In the formula (1), R1 represents hydrogen, an alkyl group, a cyano group, or an alkoxy group, and R2 represents an aryl group. The material has an ionization potential of 4.9 eV or more and 5.4 eV or less.

### TECHNICAL FIELD

The present invention relates to materials for light-emitting elements which can emit light by allowing current to flow thereto, in particular, a material having a hole injecting property, and further, a high molecular weight compound material, (also referred to as polymer) having a hole injecting property for light-emitting elements.

In recent years, a light-emitting device or a display using a light-emitting element formed with an organic material has been actively developed. The light-emitting element is manufactured by sandwiching an organic compound between a pair of electrodes. Unlike a liquid crystal display device, the light-emitting element itself emits light and does not require a light source such as a backlight. In addition, the element itself is very thin. Therefore, the light-emitting element is very advantageous in manufacturing a thin lightweight display.

In a light-emitting mechanism of the light-emitting element, it is said that electrons injected from a cathode is recombined with holes injected from an anode at a luminescent center of the organic compound to form a molecular exciton and the molecular exciton releases energy to emit light when returning to a ground state. A singlet excited state and a triplet excited state are known as excited states, and it is thought that light emission can be obtained through either of the excited states.

An organic compound layer sandwiched between electrodes often has a stacked structure. A typical example of the stacked structure is a functionally-separated stacked structure, in which thin films of a hole transporting layer, a light-emitting layer, and an electron transporting layer are sequentially stacked. By placing a layer of a highly hole transporting material on the side of an electrode serving as an anode and a layer of a highly electron transporting material on a cathode side, with a light-emitting layer in which electrons are recombined with holes sandwiched therebetween, electrons and holes can be efficiently transferred. Further, the probability of recombination of electrons and holes can be increased. Since such a structure achieves very high emission efficiency, most of light-emitting display devices which has currently been researched and developed, employ this structure (for example, Reference 1: Chihaya Adachi and three others, Japanese Journal of Applied Physics, Vol. 27, No. 2, 1988, pp. L269-L271).

As another structure, there is given a structure in which a hole injecting layer is interposed between an electrode and a hole transporting layer; or a structure in which an electron injecting layer is interposed between an electrode and an electron transporting layer. In each structure, a material having an excellent hole injecting property or a material having an excellent electron injecting property is used for the above structures. Note that a stacked structure including a layer having functions of two or more adjacent layers may be used.

Although a layer containing an organic compound typically has a stacked structure as described above, it may be a single layer or be a mixed layer. In addition, the light-emitting layer may be doped with a fluorescent pigment or the like.

Materials specific to each function have been developed. As for materials which can be used as hole injecting layers, various materials such as low molecular weight materials and high molecular weight materials have been proposed (for example, Reference 2: Japanese Patent Laid-Open No. 2000-150169 and Reference 3: UK Patent No. 2334959).

In particular, high molecular weight materials can be formed over a surface of ITO by a method such as a spin coating method or an inkjet method, which is one feature of the high molecular weight materials. Especially, an inkjet method is an important technique in manufacturing a light-emitting element, since by the inkjet method, a desired microscopic pattern can be formed by controlling a position of droplets attached on a substrate, and the inkjet method can be simply performed at low cost.

### DISCLOSURE OF INVENTION

Polyethylene dioxythiophene (PEDOT) or polyaniline of a high molecular weight material which is known as a hole injecting material is needed to be used together with acid component such as polystyrene sulphonate (PSS), and a material described in Reference 2 is needed to be used together with antimony halide. The acid component and antimony halide are known as a dopant having an electron accepting property. The acid component and antimony halide are not preferable industrially, since the former has a possibility of corrosion of an element electrode and causes poor reliability of manufactured products and the latter employs antimony which is extremely poisonous. In addition, another dopant may be used; however, needless to say, it is preferable that the number of the kinds of materials to be used is small for manufacturing a light-emitting element.

On the other hand, such dopants having an electron accepting property are not used for the material described in Reference 3. However, it is difficult to say that the material of Reference 3 has a sufficient hole injecting property, since a voltage of as high as 28V is needed for driving a light-emitting element formed using the material.

Thus, it is an object of the present invention to provide a material of a light-emitting element or a hole injecting material which has a sufficient hole injecting property, without using a dopant having an electron accepting property.

Further, it is another object of the present invention to provide a high molecular weight material for a light-emitting element or a hole injecting high molecular weight material which has a sufficient hole injecting property, without using a dopant having an electron accepting property.

Moreover, it is another object of the present invention to provide an organic compound which becomes a monomer for synthesizing a high molecular weight material of a light-emitting element or a hole injecting high molecular weight material which has a sufficient hole injecting property, without using a dopant having an electron accepting property.

Furthermore, it is another object of the present invention to provide a material for a light-emitting element or a hole injecting material which has a sufficient hole injecting property and which can suppress defects of pixels of a light-emitting element formed with the material, without using a dopant having an electron accepting property.

Additionally, there is a problem that a light-emitting element formed by an evaporation method has a low usability of materials.

In view of the above problem, it is another object of the present invention to provide a material for a light-emitting element or a hole injecting material for which a film-formation method having a high usability of materials can be used, in addition to the above-described objects.

Further, it is another object of the present invention to provide a light-emitting element with few incipient defects, which can be driven at a low voltage.

One of materials according to the present invention which can achieve the above objects is a hole injecting material, which has a polymer having a repeating unit represented by the following general formula.

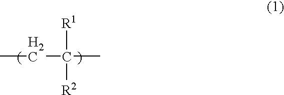

In the general formula, R¹ represents hydrogen, an alkyl group, a cyano group, or an alkoxy group, and R² represents an aryl group. The polymer has an ionization potential of 4.9 eV or more and 5.4 eV or less.

Another structure of the present invention is a hole injecting material in which an aryl group represented by R² in the general formula of the above structure has an electron donating substituent. Note that it is preferable that the electron donating substituent has a substituent constant value σ of −2.1 or more and 0.15 or less in Hammett rule.

In addition, another structure of the present invention is a hole injecting material in which an aryl group represented by R² in the general formula of the above structure has a diarylamino group.

Another material of the present invention is a hole injecting material comprising a polymer having a repeating unit represented by the following general formula (1):

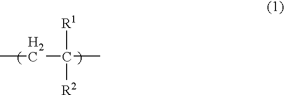

wherein R¹ represents hydrogen, an alkyl group, a cyano group, or an alkoxy group; and

wherein R² represents a group represented by the following formula (2);

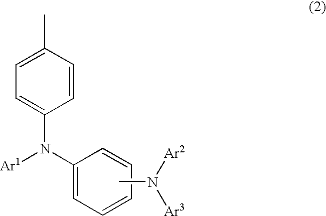

wherein each of Ar¹ to Ar³ represents an aryl group having 6 to 14 carbon atoms which may be substituted or unsubstituted.

Another material of the present invention is a hole injecting material comprising a linear polymer having a repeating unit represented by a following general formula (1):

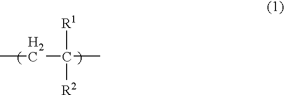

wherein R¹ represents hydrogen, an alkyl group, a cyano group, or an alkoxy group; and

wherein R² represents a group represented by the following formula (2);

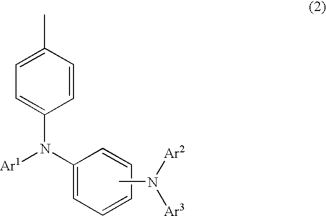

wherein each of Ar¹ to Ar³ represents an aryl group having 6 to 14 carbon atoms which may be substituted or unsubstituted.

Another material of the present invention is a hole injecting material comprising a polymer having a repeating unit represented by a following general formula (3).

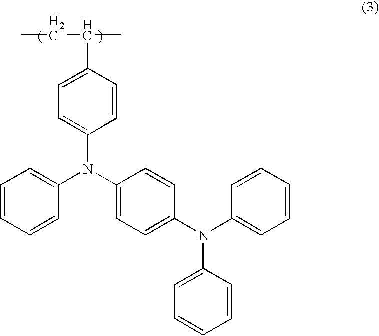

The hole injecting material preferably has a number average molecular weight of 2000 or more and 500000 or less, more preferably, a number average molecular weight of 10000 or more and 100000 or less. Further, each of the materials may have a branch, and an end group may have any group.

Another structure of the present invention is an organic compound represented by a following general formula (4),

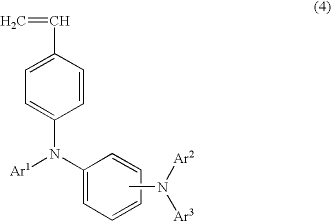

wherein each of Ar¹ to Ar³ represents an aryl group having 6 to 14 carbon atoms which may be substituted or unsubstituted.

Another structure of the present invention is an organic compound represented by a following general formula (5),

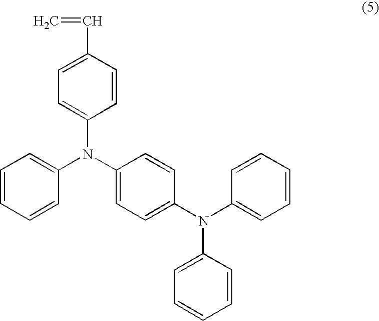

A hole injecting material of the present invention is a material for a light-emitting element or a hole injecting material which has a sufficient hole injecting property, without using a dopant having an electron accepting property.

In addition, a hole injecting material of the present invention is a high molecular weight material for a light-emitting element or a hole injecting high molecular weight material which has a sufficient hole injecting property, without using a dopant having an electron accepting property.

By using an organic compound of the present invention, a high molecular weight material for a light-emitting element or a hole injecting high molecular weight material which has a sufficient hole injecting property can be synthesized, without using a dopant having an electron accepting property.

In addition, by using a hole injecting material of the present invention, a material for a light-emitting element or a hole injecting material for which a film-formation method having a high usability of materials can be used, in addition to the above effects.

A light-emitting element of the present invention which is manufactured using a hole injecting material of the present invention is a light-emitting element with favorable characteristics which can be formed at reduced cost, since a manufacturing method of a droplet discharging method or the like typified by an inkjet method which provides a good usability of materials can be used in forming a hole injecting layer.

### BRIEF DESCRIPTION OF DRAWINGS

In the accompanying drawings:

FIG. 1 shows a light-emitting element according to an aspect of the present invention;

FIGS. 2A to 2E each show a manufacturing step of a light-emitting element according to an aspect of the present invention;

FIGS. 3A to 3C each show a manufacturing step of a light-emitting element according to an aspect of the present invention;

FIGS. 4A and 4B each show a structure of a display device as an example;

FIGS. 5A and 5B show a top view and a cross-sectional view of a light-emitting device according to an aspect of the present invention;

FIGS. 6A to 6E each show an electronic device to which the present invention can be applied;

FIGS. 7A to 7C each show a structure of a display device as an example;

FIGS. 8A to 8F each show an example of a pixel circuit of a display device;

FIG. 9 shows an example of a protective circuit of a display device;

FIG. 10 is an NMR spectrum of 4-[N-(4-diphenylaminophenyl)-N-phenyl]aminobenzaldehyde;

FIG. 11 is an NMR spectrum of 4-[N-(4-diphenylaminophenyl)-N-phenyl]aminostyrene;

FIG. 12 is an NMR spectrum of {4-[N-(4-diphenylaminophenyl)-N-phenyl]aminostyrene};

FIG. 13 shows an absorption spectrum of a PStDPA thin film;

FIG. 14 shows a current-voltage curve of an element using PStDPA as a hole injecting layer; and

FIG. 15 shows a luminescence property of an element using PStDPA as a hole injecting layer.

### BEST MODE FOR CARRYING OUT THE INVENTION

Hereinafter, the embodiment modes of the present invention will be described with reference to the accompanying drawings. The present invention can be carried out in many different modes, and it is easily understood by those skilled in the art that modes and details herein disclosed can be modified in various ways without departing from the spirit and the scope of the present invention. It should be noted that the present invention should not be interpreted as being limited to the description of the embodiment modes to be given below.

As for a pair of electrodes of a light-emitting element according to the present invention, when a voltage is applied such that a potential of one electrode thereof is higher than that of the other electrode, light is emitted. At this time, one electrode having a higher potential, is referred to as an electrode serving as an anode, and the other electrode having a lower potential, is referred to as an electrode serving as a cathode.

In the present invention, unless otherwise noted, a hole transporting layer is a layer which is formed using a substance having a hole transporting property rather than an electron transporting property, and which is located closer to an electrode serving as an anode than a light-emitting layer. An electron transporting layer is a layer which is formed using a substance having an electron transporting property rather than a hole transporting property, and which is located closer to an electrode serving as a cathode than the light-emitting layer. In addition, the hole injecting layer is a layer which is provided to be in contact with the electrode serving as an anode, and which is formed of a material with a small hole injection barrier from the electrode, and the electron injecting layer is a layer which is provided to be in contact with the electrode serving as a cathode, and which is formed of a material with a small electron injection barrier from the electrode. The adjacent layers may be one layer having the functions of the adjacent layers. Further, there is a case that a light-emitting layer has any function of the layers.

### Embodiment Mode 1

One mode of the present invention will be described.

A hole injecting material of the present invention is a polymer having a repeating unit represented by the following general formula (1). In the formula (1), R¹ represents hydrogen, an alkyl group, a cyano group, or an alkoxy group, and R² represents an aryl group. In the polymer with such a structure, a polymer having ionization potential of 4.9 eV or more and 5.4 eV or less is a polymer which can accept holes smoothly from a metal, and can be used favorably as a hole injecting material.

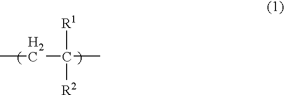

In the general formula, the aryl group represented by R² preferably has an electron donating substituent, and it is preferable that the electron donating substituent has a substituent constant value σ of −2.1 or more and 0.15 or less in Hammett rule. This is because it can be favorably used as a hole injecting material in this case without using an electron donating dopant.

The aryl group represented by R² of the general formula is preferably an aryl group having a diarylamine group in order to obtain a polymer which can be preferably used as a hole injecting material particularly, without using a dopant having an electron donating property.

In particular, a group of an aryl group with a diarylamine group, which is represented by the following general formula (2) is extremely preferable.

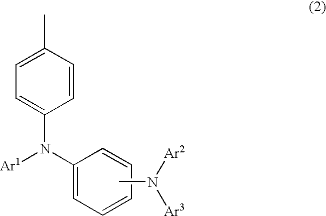

In the above general formula, Ar¹ to Ar³ independently represents an aryl group having 6 to 14 carbon atoms, and the aryl group may be substituted or unsubstituted. Ar² and A3 may condense with each other.

A polymer having such a structure can be a polymer which has ionization potential of 4.9 eV or more and 5.4 eV or less, and which can accept holes smoothly from a metal. It can be favorably used as a hole injecting material.

As for the hole injecting material of the present invention represented by the above general formula (1), a representative example is shown below. The hole injecting material of the present invention is not limited to this example.

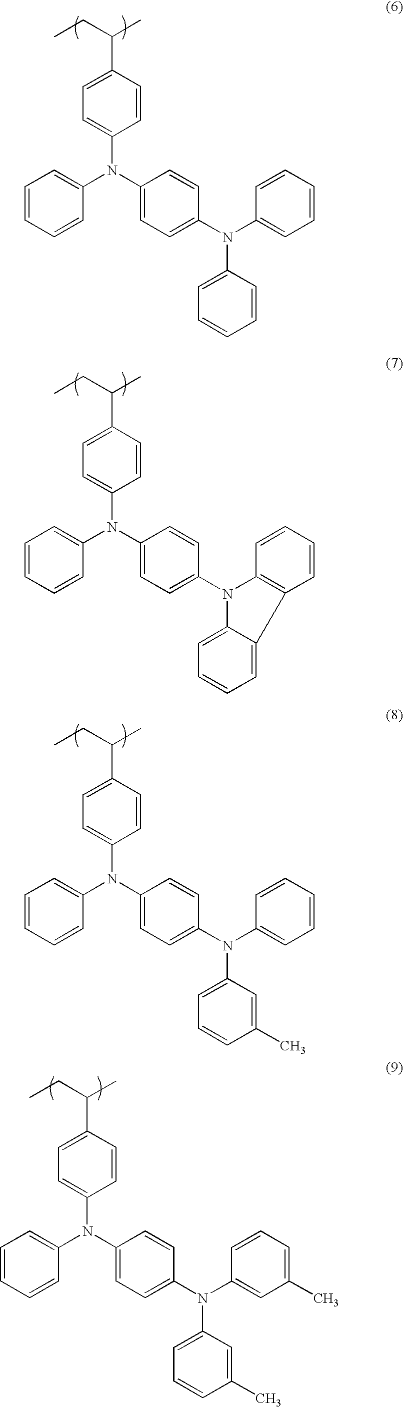

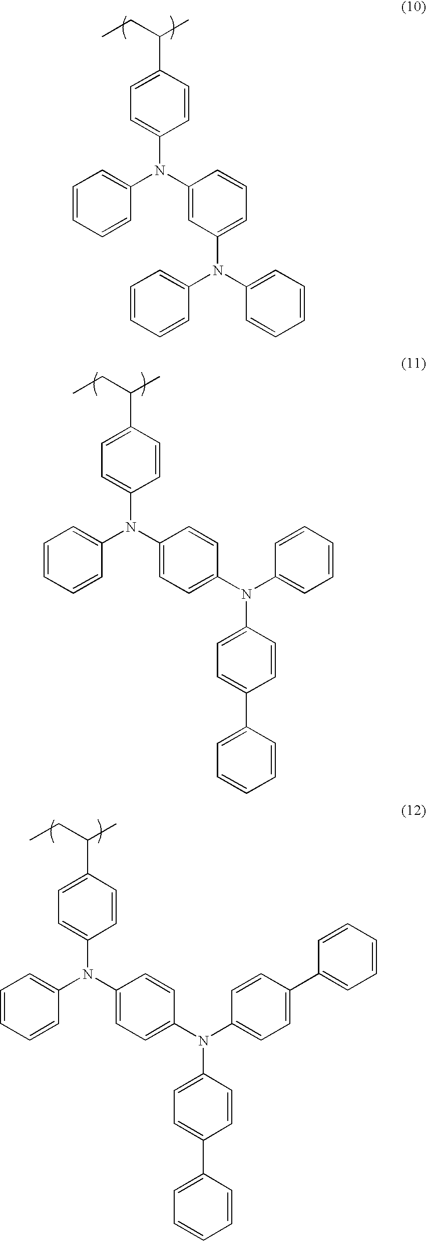

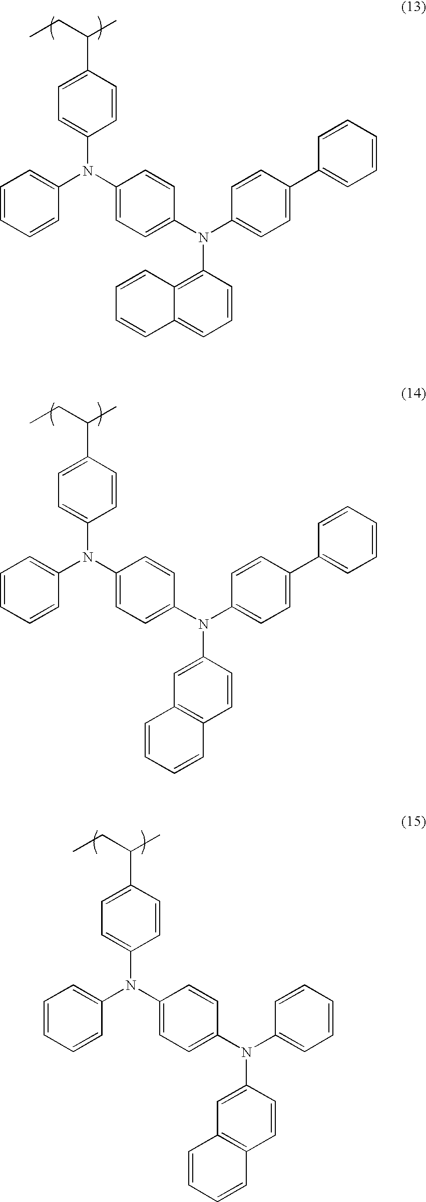

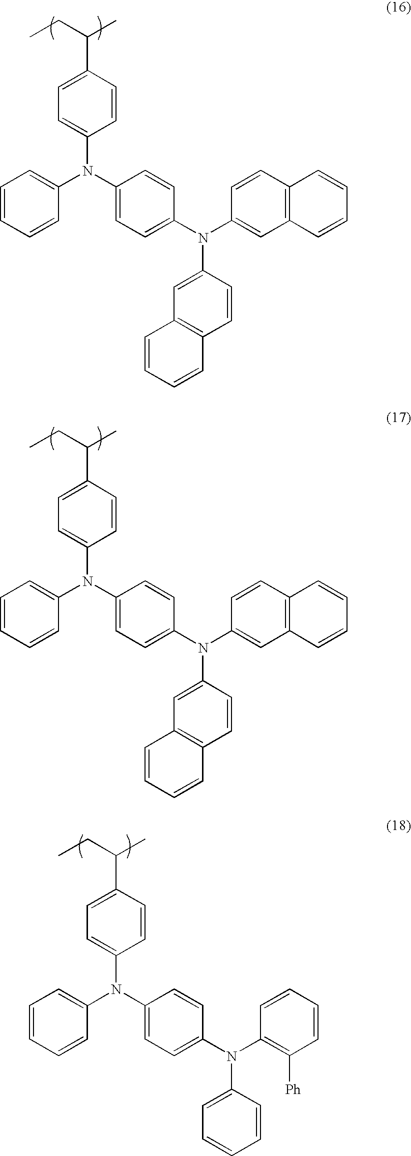

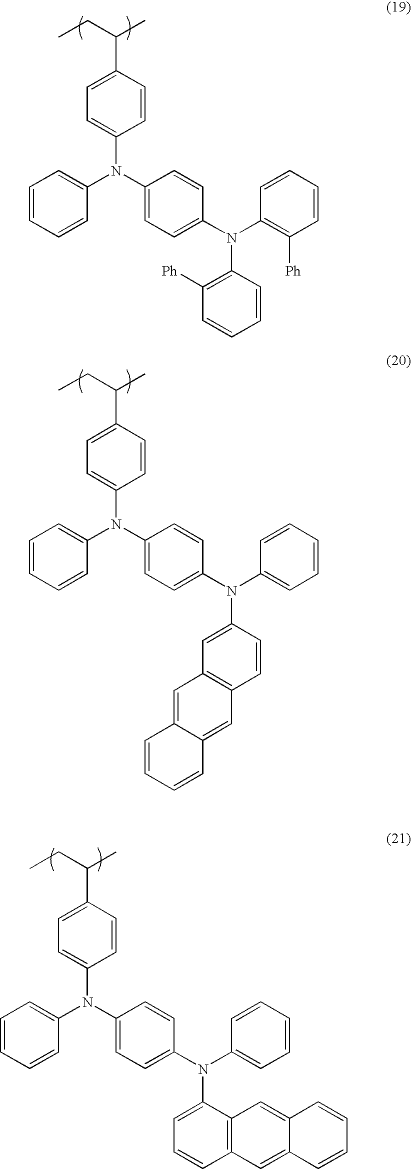

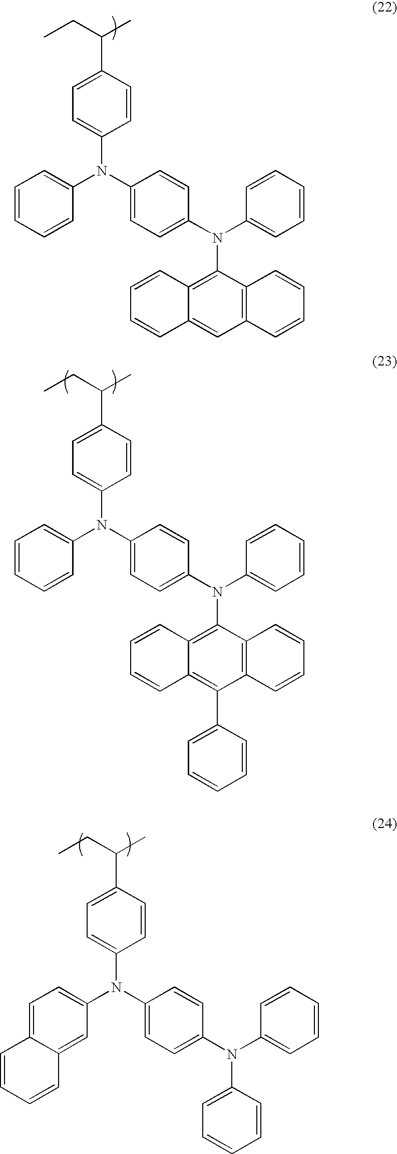

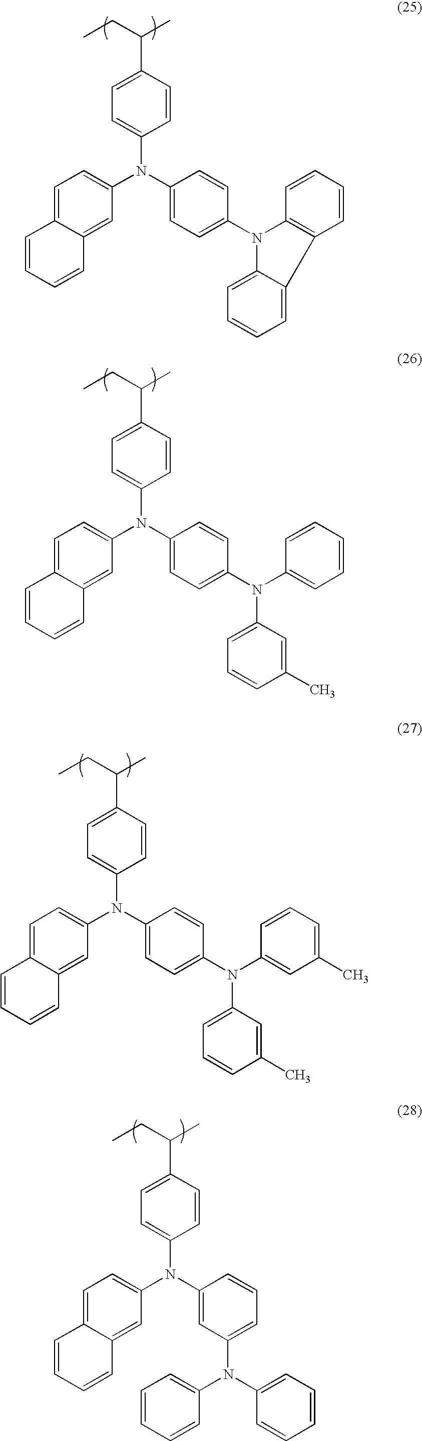

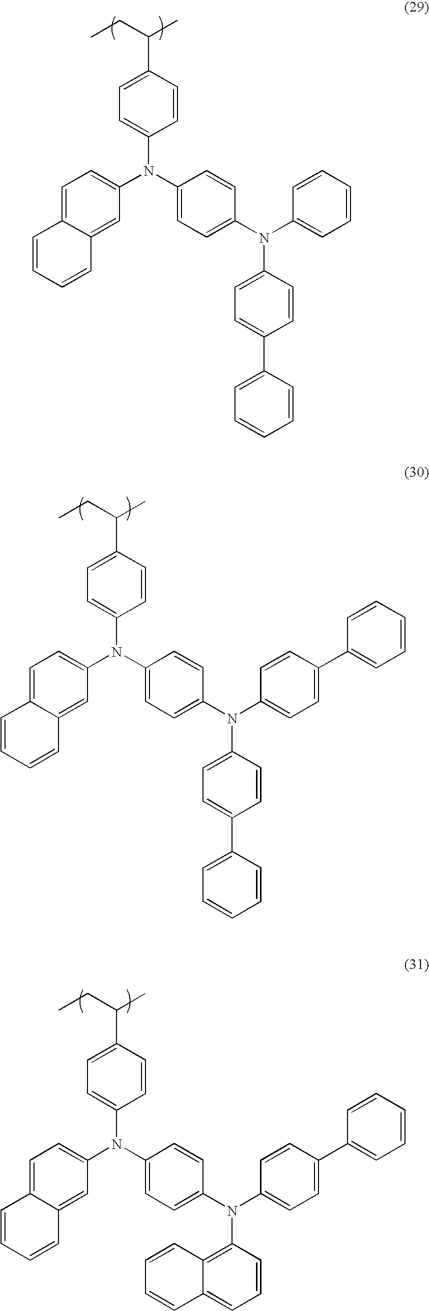

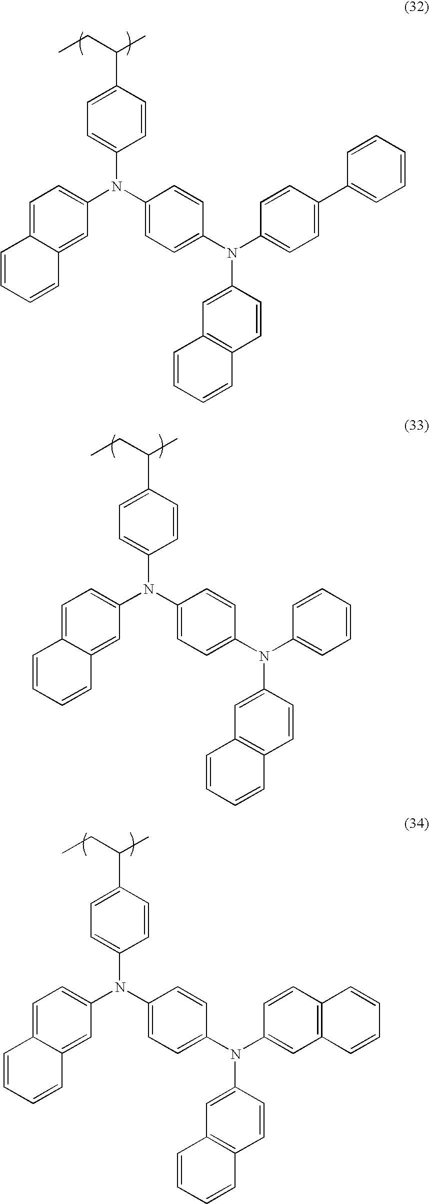

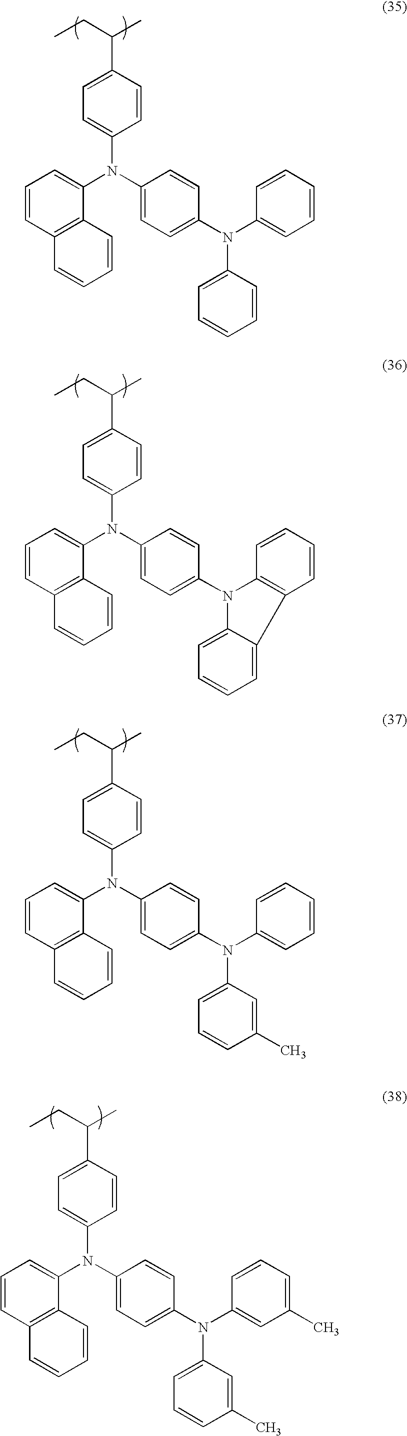

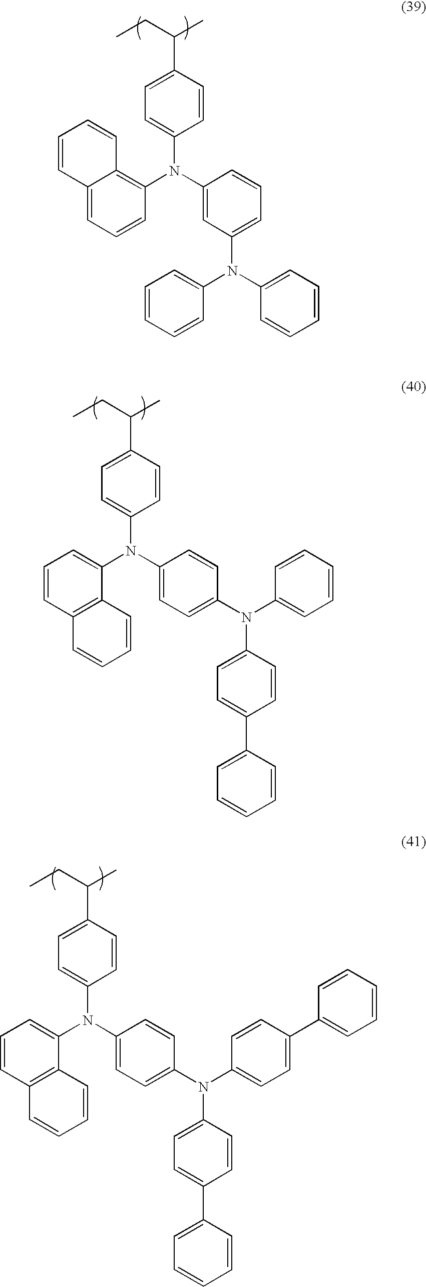

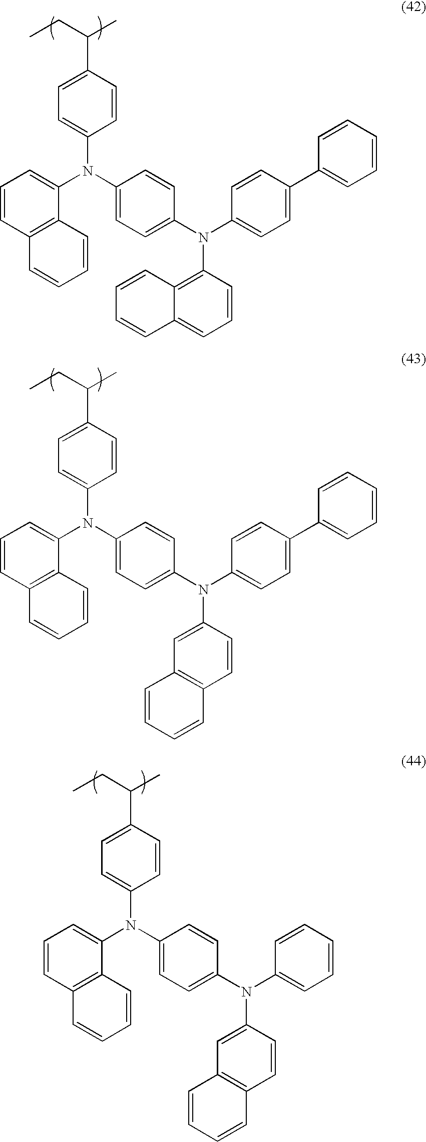

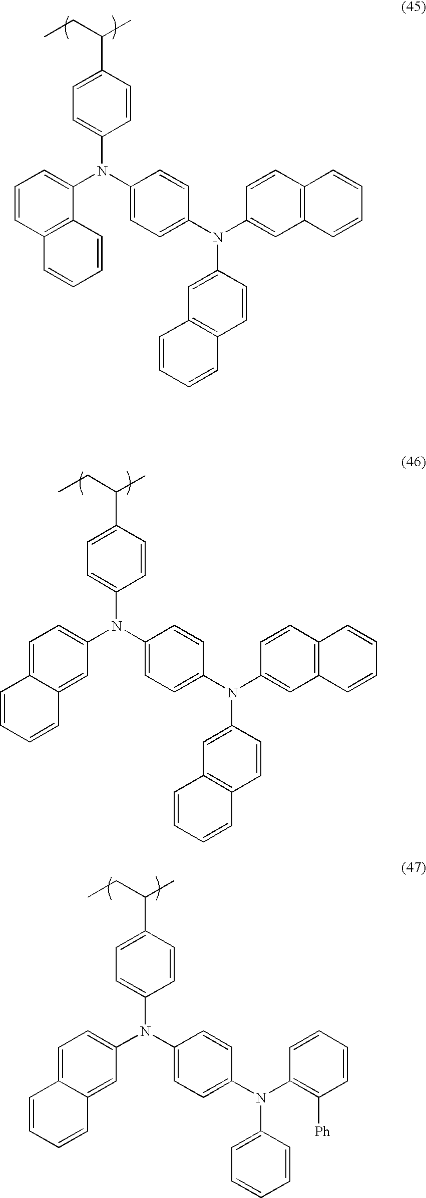

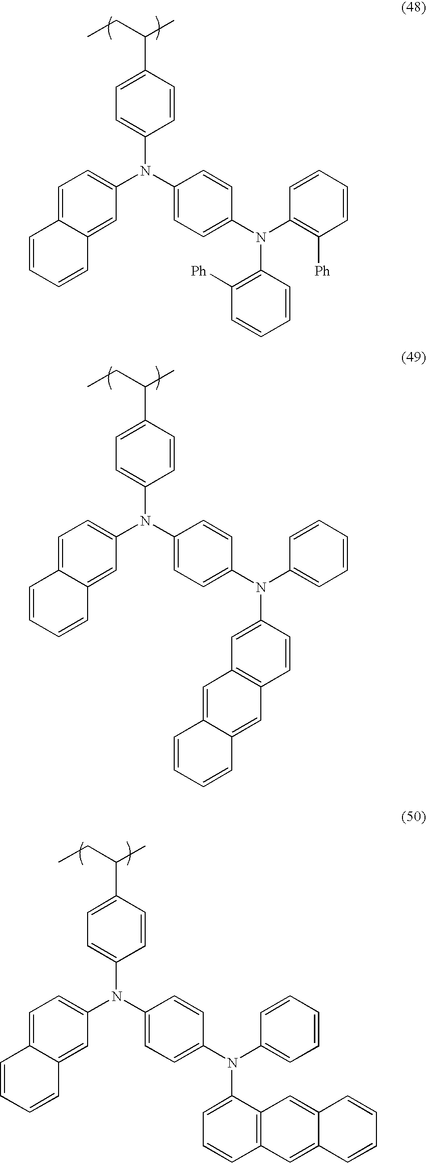

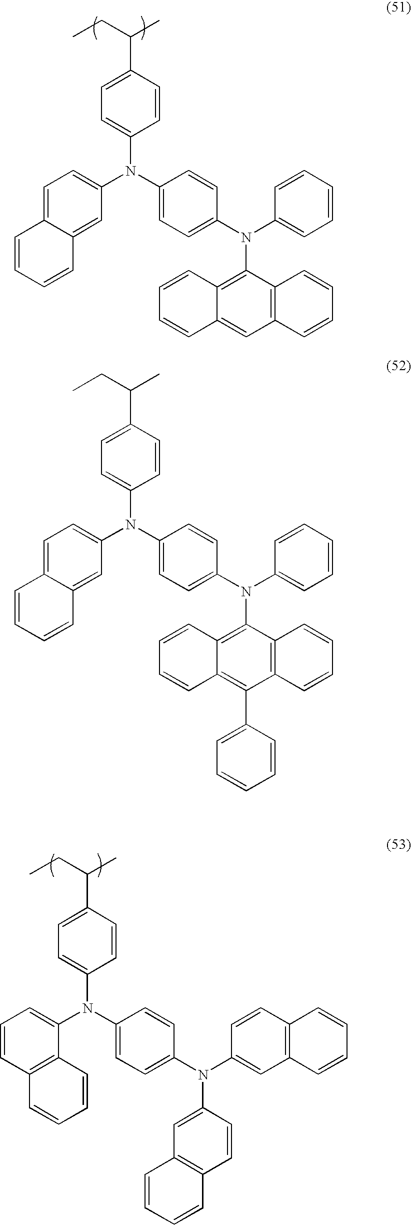

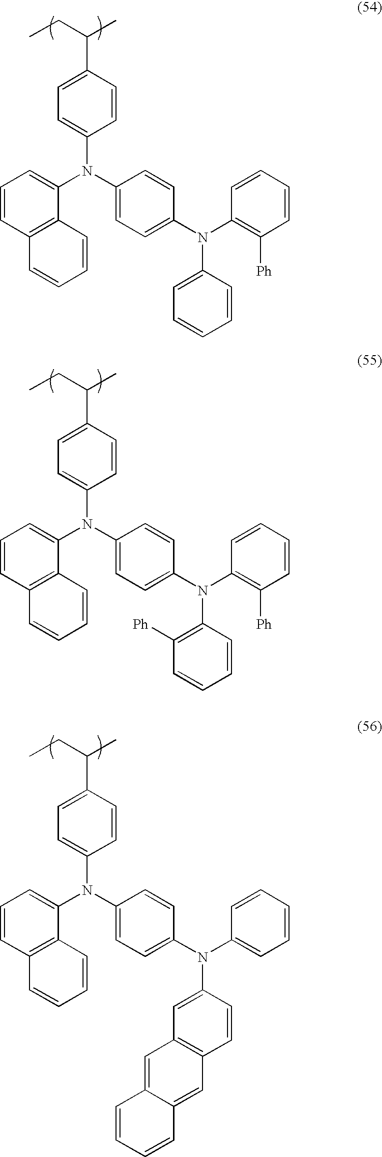

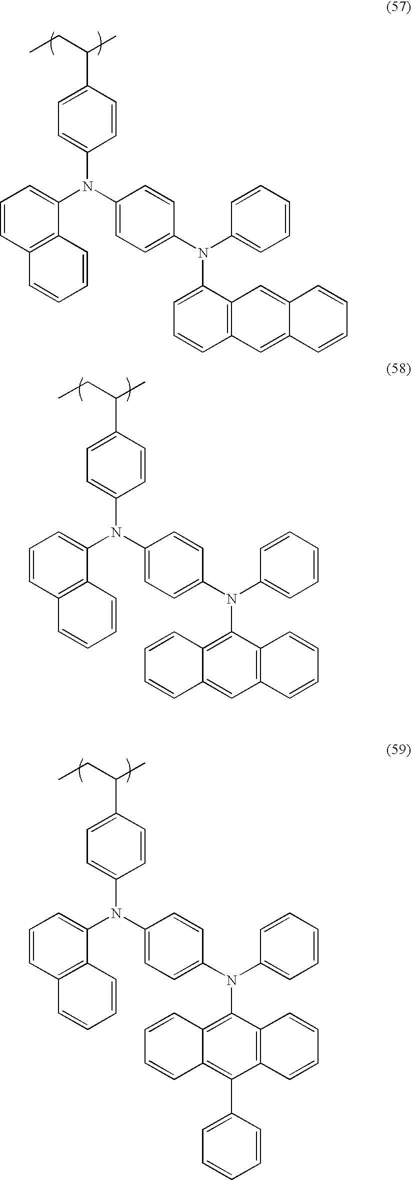

There is no limitation on an end group of the hole injecting material of the present invention and further, no limitation on a synthesis method thereof.

In addition, the hole injecting material of the present invention may be a material in which two or more types of groups represented by the above formula (2) are mixed in each molecule, or may be a material of a copolymer in which two or more types of groups represented by the above formula (2) are mixed in one molecule.

The extent of branching influences crystallinity, density or stiffness; however, crystallinity, density or stiffness can be set appropriately with a known method by a user in consideration of reaction, since the influences on the hole injecting property is small.

The number average molecular weight of the polymer is preferably in the range of 2000 to 500000, more preferably, the number average molecular weight is 10000 or more and 100000 or less.

A hole injecting material made of such a polymer has a sufficient hole injecting property without using a dopant having an electron accepting property, and the hole injecting material itself can be used as a material for forming a hole injecting layer of a light-emitting element.

A hole injecting material of the present invention made of such a polymer is used as a material for forming a hole injecting layer of a light-emitting element. Thus, since a manufacturing method such as a droplet-discharging method typified by an inkjet method which provides a good usability of materials can be used in forming a hole injecting layer, a light-emitting element with favorable characteristics can be formed at reduced cost.

In other words, a light-emitting element having favorable characteristics can be manufactured at reduced cost, without corrosion of an electrode, the use of extremely poisonous substance, and the rise of driving voltage.

Further, since a hole injecting material made of such a polymer for a light-emitting element can be formed by a droplet-discharging method represented by an inkjet method, the usability of materials can be increased drastically.

### Embodiment Mode 2

As one mode of the present invention, an organic compound of a monomer of the present invention which is used for synthesizing a hole injecting material of the present invention, as shown in Embodiment Mode 1, will be described.

A monomer of the present invention is an organic compound represented by the following general formula (4). In the formula, Ar¹ to Ar³ individually represent an aryl group having 6 to 14 carbon atoms, and the aryl group may be substituted or unsubstituted. In addition, Ar² and Ar³ may condense with each other.

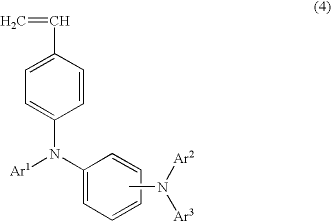

By polymerizing the organic compound represented by the above general formula (4), a hole injecting material of a polymer can be synthesized, which is one mode of the present invention and is represented by the following general formula (1).

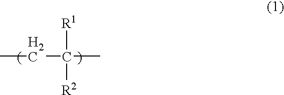

However, in the formula, R¹ represents hydrogen, an alkyl group, a cyano group, or an alkoxy group, and R² represents a group represented by the general formula (2).

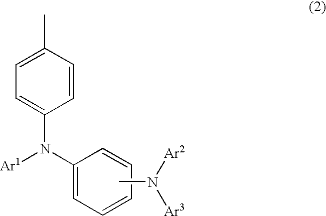

In the above formula, Ar¹ to Ar³ individually represent an aryl group having 6 to 14 carbon atoms, and the aryl group may be substituted or unsubstituted. In addition, Ar² and Ar³ may condense with each other.

By polymerizing an organic compound of the present invention represented by the above formula (4) as a monomer, a hole injecting material of the present invention as shown in Embodiment Mode 1 can be synthesized, which a polymer having a sufficient hole injecting property without using an electron accepting dopant, and capable of forming a hole injecting layer of a light-emitting element by itself.

In addition, by copolymerizing a monomer mixture which is shown in the above formula (4) and is a mixture of at least two types of organic compounds having different structures, a hole injecting material of the present invention which is a polymer with two or more types of substituents represented by R² in the above formula (1) can be synthesized. With copolymerization of different types of monomers, the design of crystallinity, molecular weight, glass-transition temperature, and the like can be thought to be conducted easily.

### Embodiment Mode 3

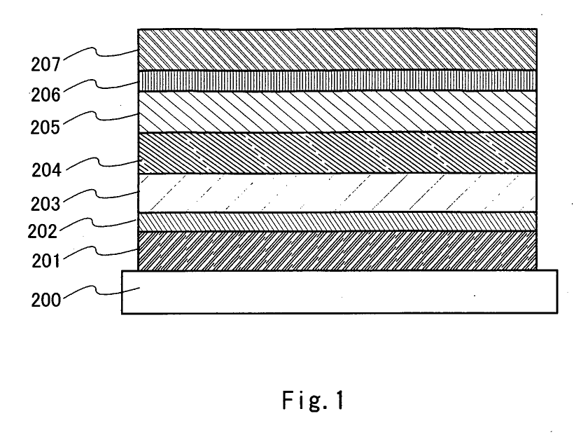

A light-emitting element of the present invention will be described as one mode of the present invention. FIG. 1 shows a structure of the light-emitting element of the present invention as one example.

A hole injecting layer in the light-emitting element of the present invention is formed by a hole injecting material of a polymer having a repeating unit represented by the following general formula (1). In the formula (1), R¹ represents hydrogen, an alkyl group, a cyano group, or an alkoxy group, and R² represents any one of groups represented by the following general formula (2).

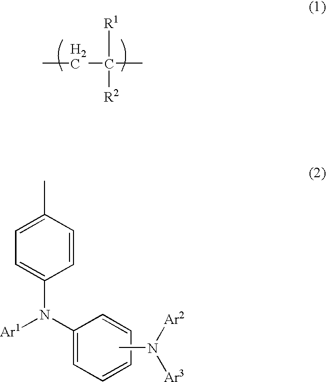

In the above formula, Ar¹ to Ar³ individually represent an aryl group having 6 to 14 carbon atoms, and the aryl group may be substituted or unsubstituted. In addition, Ar¹ and Ar² may condense with each other.

The mode of the light-emitting element using a hole injecting material of the present invention as a hole injecting layer will be described with reference to FIG. 1.

FIG. 1 shows a structure of a light-emitting element in which an organic layer including a hole injecting layer 202, a hole transporting layer 203, a light-emitting layer 204, an electron transporting layer 205, and an electron injecting layer 206 is interposed between a first electrode 201 and a second electrode 207 over a substrate 200. The hole injecting layer 202 includes a hole injecting material of the present invention represented by any one of the general formulas (1) and (3) and the structural formulas (6) to (59).

In the light-emitting element, holes from the first electrode 201 are injected into the hole injecting layer 202 made of a hole injecting material of the present invention, and the holes are recombined with electrons injected from the second electrode 207, so that a light-emitting substance included in the light-emitting layer 204 is excited. Then, when the excited light-emitting substance returns to a ground state, light is generated and emitted.

In this manner, when a hole injecting material of the present invention is used for the hole injecting layer 202, holes from the first electrode 201 is injected into the organic layer smoothly, since the injection barrier of holes from the first electrode 201 is small. As a result, the driving voltage can be reduced. In addition, there is no need to use electron donating dopant such as acid component and antimony halide, and there is no concern of electrode damages or environmental pollution. In the light-emitting element of this embodiment mode, the first electrode 201 serves as an anode and the second electrode 207 serves as a cathode.

There is no particular limitation on the light-emitting layer 204; however, a layer serving as the light-emitting layer 204 has two modes roughly. The one is a host-guest type layer which includes a dispersed light-emitting material in a layer formed of a material (host material) having a larger energy gap than an energy gap of a light-emitting substance which becomes a luminescent center, while the other is a layer in which a light-emitting layer is made of a light-emitting material only. The former is preferable, since concentration quenching is difficult to be caused. As the light-emitting substance to be a luminescent center, the following can be employed; 4-dicyanomethylene-2-methyl-6-(1,1,7,7-tetramethyljulolidyl-9-enyl)-4H-pyran (DCJT); 4-dicyanomethylene-2-t-butyl-6-(1,1,7,7-tetramethyl-julolidine-9-enyl)-4H-pyran; periflanthene; 2,5-dicyano-1,4-bis (10-methoxy-1,1,7,7-tetramethyljulolidine-9-enyl)benzene; N,N′-dimethylquinacridone (DMQd); coumarin 6; coumarin 545T; tris (8-quinolinolato)aluminum (Alq₃); 9,9′-bianthryl; 9,10-diphenylanthracene (DPA); 9,10-bis(2-naphthyl)anthracene (DNA); 2,5,8,11-tetra-t-butylperylene (TBP); or the like. As the material to be a host material in the case of forming the layer in which the light-emitting material is diffused, the following can be used; an anthracene derivative such as 9,10-di(2-naphtyl)-2-tert-butylanthracene (t-BuDNA); a carbazole derivative such as 4,4′-bis(N-carbazolyl)biphenyl (CBP); or a metal complex such as tris(8-quinolinolato)aluminum (Alq₃), tris(4-methyl-8-quinolinolato)aluminum (Almq₃); bis(10-hydroxybenzo[h]-quinolinato)beryllium (BeBq₂); bis(2-methyl-8-quinolinolato)-4-phenylphenolato-aluminum (BAlq); bis[2-(2-hydroxyphenyl)pyridinato]zinc (Znpp₂); or bis[2-(2-hydroxyphenyl)benzoxazolate]zinc (ZnBOX). As the material which can constitute the light-emitting layer 204 only with a light-emitting substance, tris(8-quinolinolato)aluminum (Alq₃), 9,10-bis(2-naphtyl)anthracene (DNA), or bis(2-methyl-8-quinolinolato)-4-phenylphenolato-aluminum (BAlq) or the like can be used.

Further, there is no limitation on the first electrode 201; however, the first electrode 201 is preferably formed with a substance having a high work function when it serves as an anode. Specifically, in addition to indium tin oxide (ITO), indium tin oxide containing silicon (ITSO), and indium oxide including zinc oxide (IZO), gold (Au), platinum (Pt), nickel (Ni), tungsten (W), chromium (Cr), molybdenum (Mo), iron (Fe), cobalt (Co), copper (Cu), palladium (Pd), or the like can be used. The first electrode 201 can be formed by, for example, a sputtering method or an evaporation method.

Further, although there is no particular limitation on the second electrode 207, the second electrode 207 is preferably formed of a material that has a low work function when the second electrode 207 functions as a cathode as in this embodiment mode. Specifically, aluminum containing an alkali metal or an alkali-earth metal such as lithium (Li) or magnesium, or the like can be used. The second electrode 207 can be formed by, for example, a sputtering method or an evaporation method.

In order to extract emitted light to the outside, one of or both the first electrode 201 and the second electrode 207 be an electrode containing a material such as indium tin oxide or an electrode formed to have a thickness of several to several tens nm so as to transmit visible light.

In addition, a hole transporting layer 203 need not be provided between the first electrode 201 and the light-emitting layer 204 as shown in FIG. 1. Here, a hole transporting layer 203 is a layer that has a function of transporting holes injected from the first electrode 201 into the light-emitting layer 204.

There is no limitation on the hole transporting layer 203, and it is possible to use a layer formed with the use of, for example, an aromatic amine compound (that is, compound including a bond of a benzene ring-nitrogen) such as 4,4′-bis[N-(1-naphthyl)-N-phenyl-amino]-biphenyl (α-NPD), 4,4′-bis[N-(3-methylphenyl)-N-phenyl-amino]-biphenyl (TPD), 4,4′,4″-tris(N,N-diphenyl-amino)-triphenylamine (TDATA), or 4,4′, 4″-tris[N-(3-methylphenyl)-N-phenyl-amino]-triphenylamine (MTDATA).

Moreover, the hole transporting layer 203 may be a multilayer which is formed by combining two or more layers which are formed of the above substances.

Further, an electron transporting layer 205 may be provided between the second electrode 207 and the light-emitting layer 204 as shown in FIG. 1, or not. Here an electron transporting layer is a layer that has a function of transporting electrons injected from the second electrode 207 into the light-emitting layer 204. The electron transporting layer 205 is provided to keep the second electrode 207 away from the light-emitting layer 204 in this way; therefore, quenching of luminescence due to a metal can be prevented.

There in no limitation on the electron transporting layer 205, and it is possible to use a layer formed using, for example, a metal complex including a quinoline skeleton or a benzoquinoline skeleton, such as tris(8-quinolinolato)aluminum (Alq₃), tris(5-methyl-8-quinolinolato)aluminum (Almq₃), bis(10-hydroxybenzo[h]quinolinato) beryllium (BeBq₂), or bis(2-methyl-8-quinolinolato)-4-phenylphenolato-aluminum (BAlq). In addition, a layer formed using, for example, a metal complex including a oxazole-based ligand or a thiazole-based ligand such as bis[2-(2-hydroxyphenyl)-benzoxazolato]zinc (Zn(BOX)₂) or bis[2-(2-hydroxyphenyl)-benzothiazolato]zinc (Zn(BTZ)₂), may be used. Further, a layer formed with the use of 2-(4-biphenylyl)-5-(4-tert-butylphenyl)-1,3,4-oxadiazole (PBD), 1,3-bis[5-(p-tert-butylphenyl)-1,3,4-oxadiazole-2-yl]benzene (OXD-7), 3-(4-tert-butylphenyl)-4-phenyl-5-(4-biphenylyl)-1,2,4-triazole (TAZ), 3-(4-tert-butylphenyl)-4-(4-ethylphenyl)-5-(4-biphenylyl)-1,2,4-triazole (p-EtTAZ), bathophenanthroline (BPhen), bathocuproin (BCP), or the like may be used.

Moreover, the electron transporting layer 205 may be a multilayer combining two or more layers which are formed of the above substances.

Further, an electron injecting layer 206 need not be provided between the second electrode 207 and the electron transporting layer 205, unlike in FIG. 1. Here, an electron injecting layer is a layer that has a function of assisting injection of electrons from an electrode serving as a cathode to the electron transporting layer 205. It is to be noted that injection of electrons into a light-emitting layer may be assisted by providing an electron injecting layer between an electrode serving as a cathode and the light-emitting layer when an electron transporting layer is not provided.

There is no limitation on the electron injecting layer 206, and it is possible to use a layer formed with the use of, for example, a compound of an alkali metal or an alkali-earth metal such as lithium fluoride (LiF), cesium fluoride (CsF), or calcium fluoride (CaF₂). In addition, a layer in which a highly electron transportable material such as Alq₃ or 4,4-bis(5-methylbenzoxazol-2-yl)stilbene (BzOs) is mixed with an alkali metal or an alkali-earth metal such as magnesium or lithium can also be used as the electron injecting layer 206.

In the above-described light-emitting element, each of the hole transporting layer 203, the light-emitting layer 204, the electron transporting layer 205, and the electron injecting layer 206 may be formed by any method, for example, an evaporation method, an inkjet method or a coating method. In addition, the first electrode 201 and the second electrode 207 may be formed by any method, for example, a sputtering method or an evaporation method.

The light-emitting element having the above structure is a light-emitting element which can be driven at a low voltage, since the hole injecting layer 202 is formed with a hole injecting material of the present invention, the injection barrier of holes from the first electrode 201 is small and holes are injected smoothly into the organic layer from the first electrode 201. In addition, a light-emitting element of the present invention need not to use electron donating dopant such as acid component and antimony halide as a material of the hole injecting layer, and thus, there is no concern of electrode damages or environmental pollution. Further, since the light-emitting element of the present invention employs a hole injecting material 202 of the present invention which can adopt a formation method with high usability of materials, as a material of the hole injecting layer 202, the light-emitting element of the present invention has an advantage in cost.

### Embodiment Mode 4

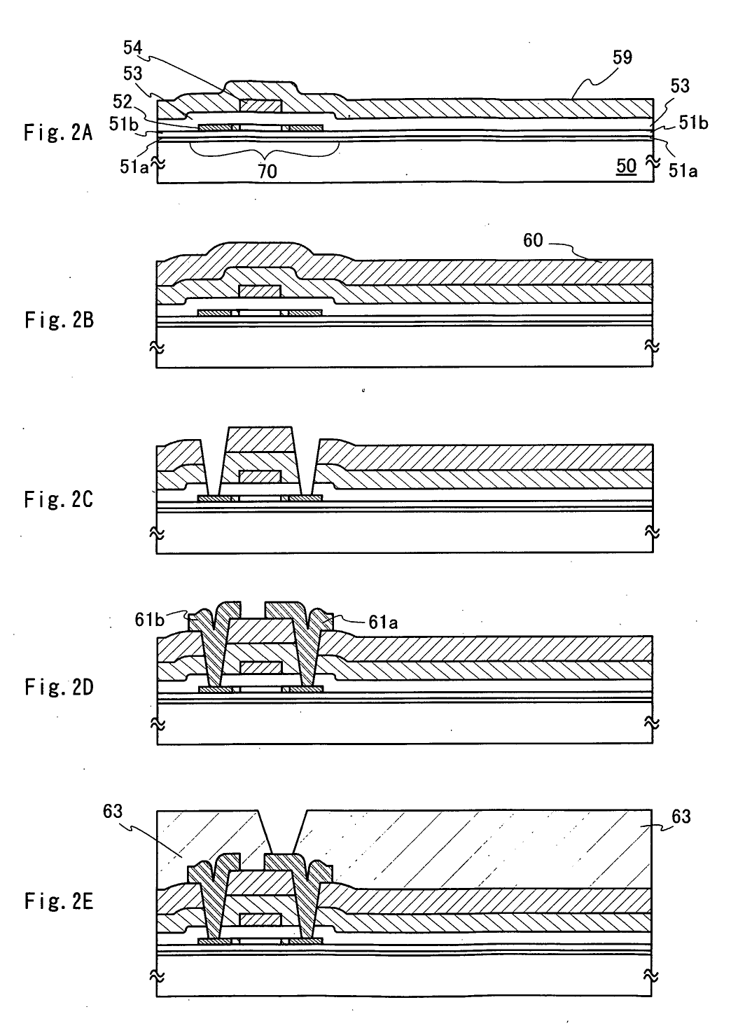

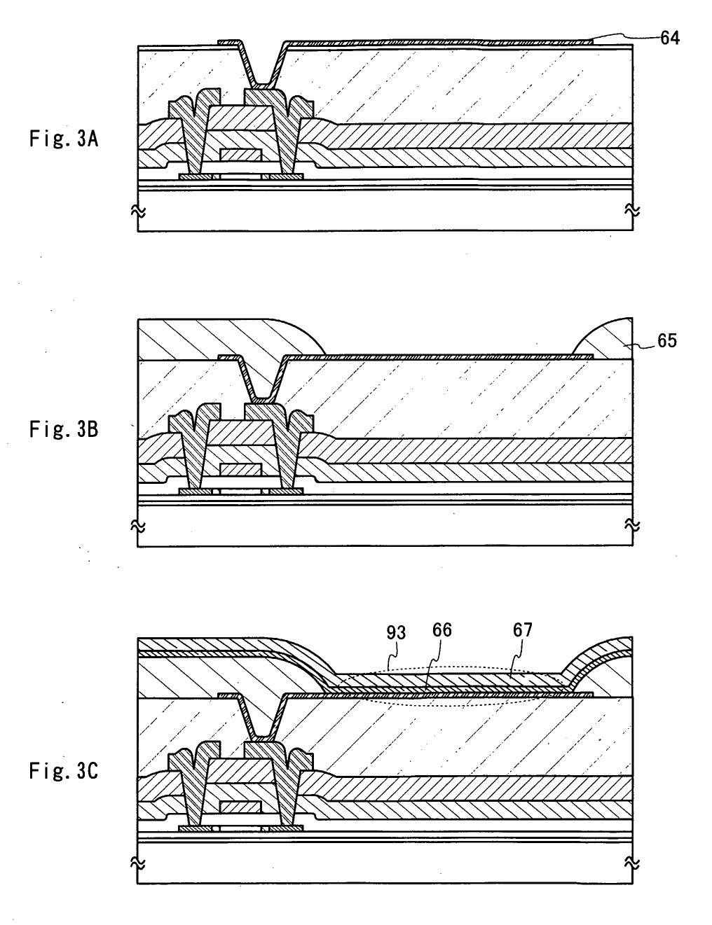

This embodiment mode describes a display device of the present invention while showing its manufacturing method with reference to FIGS. 2A to 2E and 3A to 3C. Although this embodiment mode shows an example of manufacturing an active matrix display device, it is natural that a light-emitting device of the present invention is also applicable for a passive matrix display device.

First, a first base insulating layer 51a and a second base insulating layer 51b. are formed over a substrate 50, and then a semiconductor layer is formed over the second base insulating layer 51b (FIG. 2A).

As a material of the substrate 50, glass, quartz, plastic (such as polyimide, acrylic, polyethylene terephthalate, polycarbonate, polyacrylate, or polyethersulfone), or the like can be used. These substrates may be used after being polished by CMP or the like as necessary. In this embodiment mode, a glass substrate is used.

The first base insulating layer 51a and the second base insulating layer 51b are provided in order to prevent an element which adversely affects the characteristic of the semiconductor film such as alkali metal or alkali-earth metal in the substrate 50 from diffusing into the semiconductor layer. As the material of these base insulating layers, silicon oxide, silicon nitride, silicon oxide containing nitrogen, silicon nitride containing oxygen, or the like can be used. In this embodiment mode, the first base insulating layer 51a is formed with silicon nitride, and the second base insulating layer 51b is formed with silicon oxide. Although the base insulating layer is formed to have a two-layer structure including the first base insulating layer 51a and the second base insulating layer 51b in this embodiment mode, the base insulating layer may be formed to have a single-layer structure or a multilayer structure including two or more layers. The base insulating layer is not necessary when the diffusion of the impurity from the substrate does not lead to a significant problem.

The semiconductor layer formed subsequently is obtained by crystallizing an amorphous silicon film with a laser beam in this embodiment mode. The amorphous silicon film is formed to have a thickness of 25 to 100 nm (preferably 30 to 60 nm) over the second base insulating layer 51b by a known method such as a sputtering method, a low-pressure CVD method, or a plasma CVD method. After that, a heat treatment is conducted for 1 hour at 500° C. for dehydrogenation.

Next, the amorphous silicon film is crystallized with a laser irradiation apparatus to form a crystalline silicon film. In this embodiment mode, an excimer laser is used at the laser crystallization in this embodiment mode. After the emitted laser beam is shaped into a linear beam spot using an optical system, the amorphous silicon film is irradiated with the linear beam spot. Thus, the crystalline silicon film is obtained and is used as the semiconductor layer.

Alternatively, the amorphous silicon film can be crystallized by another method such as a method in which the crystallization is conducted only by a heat treatment or a method in which a heat treatment is conducted using a catalyst element for inducing the crystallization. As the element for inducing the crystallization, nickel, iron, palladium, tin, lead, cobalt, platinum, copper, gold, or the like is given. By using such an element, the crystallization is conducted at lower temperature in shorter time than the crystallization only by the heat treatment; therefore, the damage to the glass substrate is small. In the case of crystallization only by the heat treatment, a quartz substrate which can resist the high temperature is preferably used as the substrate 50.

Subsequently, a small amount of impurity elements are added to the semiconductor layer as necessary in order to control the threshold, which is so-called channel doping. In order to obtain the required threshold, an impurity showing N-type or P-type (such as phosphorus or boron) is added by an ion-doping method or the like.

After that, as shown in FIG. 2A, the semiconductor layer is patterned into a predetermined shape so that an island-shaped semiconductor layer 52 is obtained. The patterning is conducted by etching the semiconductor layer using a mask. The mask is formed in such a way that a photo resist is applied to the semiconductor layer and the photo resist is exposed and baked, so that a resist mask having a desired mask pattern is formed over the semiconductor layer.

Next, a gate insulating layer 53 is formed so as to cover the semiconductor layer 52. The gate insulating layer 53 is formed to have a thickness of 40 to 150 nm with an insulating layer containing silicon by a plasma CVD method or a sputtering method. In this embodiment mode, silicon oxide is used.

Then, a gate electrode 54 is formed over the gate insulating layer 53. The gate electrode 54 may be formed with an element selected from tantalum, tungsten, titanium, molybdenum, aluminum, copper, chromium, or niobium, or may be formed with an alloy material or a compound material which contains the above element as its main component. Further, a semiconductor film typified by a polycrystalline silicon film doped with an impurity element such as phosphorus may be used. Ag—Pd—Cu alloy may also be used.

Although the gate electrode 54 is formed as a single layer in this embodiment mode, the gate electrode 54 may have a multilayer structure including two or more layers of, for example, tungsten as a lower layer and molybdenum as an upper layer. Even in the case of forming the gate electrode to have the multilayer structure, the above-mentioned material may be used. The combination of the above materials may also be selected appropriately. The gate electrode 54 is processed by etching with the use of a mask formed with a photo resist.

Subsequently, impurities are added to the semiconductor layer 52 at high concentration using the gate electrode 54 as the mask. By this step, a thin film transistor 70 including the semiconductor layer 52, the gate insulating layer 53, and the gate electrode 54 is formed.

The manufacturing process of the thin film transistor is not limited especially, and may be modified appropriately so that a transistor having a desired structure can be manufactured.

Although this embodiment mode employs a top-gate thin film transistor using the crystalline silicon film obtained by the laser crystallization, a bottom-gate type thin film transistor using an amorphous semiconductor film can also be applied to a pixel portion. Not only silicon but also silicon germanium can be used for the amorphous semiconductor. In the case of using silicon germanium, the concentration of germanium preferably ranges from approximately 0.01 to 4.5 atomic %.

Moreover, a microcrystal semiconductor (semi-amorphous semiconductor) film which includes crystal grains each having a diameter of 0.5 to 20 nm in the amorphous semiconductor may also be used. The microcrystal having the crystal with a diameter of 0.5 to 20 nm is also referred to as a so-called microcrystal (μc).

Semi-amorphous silicon (also referred to as SAS), which belongs to the semi-amorphous semiconductor, can be obtained by decomposing a silicon source gas by glow discharging. As typical silicon source gases, SiH₄ is given. Besides, Si2H6, SiH₂Cl₂, SiHCl₃, SiCl₄, SiF₄, or the like can be used. By using the silicon source gas after diluting the silicon source gas with hydrogen or hydrogen and one or plural kinds of a rare gas selected from helium, argon, krypton, or neon, SAS can be easily formed. The silicon source gas is preferably diluted with the dilution ratio of 1:10 to 1:1000. The reaction to form the film by the decomposition by glow discharging may be conducted at the pressure ranging from 0.1 to 133 Pa. The electric power for forming the glow discharging may be supplied at high frequency in the range of 1 to 120 MHz, preferably 13 to 60 MHz. The substrate heat temperature is preferably 300° C. or less, preferably in the range of 100 to 250° C.

The raman spectrum of thus formed SAS shifts to the side of lower wavenumber than 520 cm⁻¹. According to X-ray diffraction, diffraction peaks of a silicon crystal lattice are observed at (111) and (220). As a terminating agent of a dangling bond, hydrogen or halogen is added at least 1 atomic %. As the impurity element in the film, the impurity in the air such as oxygen, nitrogen, or carbon is desirably 1×10²⁰ cm⁻¹ or less, and in particular, the concentration of oxygen is 5×10¹⁹/cm³ or less, preferably 1×10¹⁹/cm³ or less. The electron field-effect mobility of a TFT manufactured with this film is μ=1 to 10 cm²/Vsec.

This SAS may be used after being crystallized further with a laser beam.

Subsequently, an insulating film (film containing hydrogen) 59 is formed with silicon nitride to cover the gate electrode 54 and the gate insulating layer 53. After forming the insulating film (film containing hydrogen) 59, a heat treatment is conducted for approximately 1 hour at 480° C. so as to activate the impurity element and to hydrogenate the semiconductor layer 52.

Subsequently, a first interlayer insulating layer 60 is formed to cover the insulating film (film containing hydrogen) 59. As a material for forming the first interlayer insulating layer 60, silicon oxide, acrylic, polyimide, siloxane, a low-k material, or the like is preferably used. In this embodiment mode, a silicon oxide film is formed as the first interlayer insulating layer (FIG. 2B). In this specification, siloxane is a material whose skeletal structure includes a bond of silicon and oxygen and which has an organic group containing at least hydrogen (such as an alkyl group or an aryl group), a fluoro group, or the organic group containing at least hydrogen and the fluoro group as the substituent.

Next, contact holes that reach the semiconductor layer 52 are formed. The contact holes can be formed by etching with a resist mask until the semiconductor layer 52 is exposed. Either wet etching or dry etching can be applied. The etching may be conducted once or multiple times depending on the condition. When the etching is conducted multiple times, both of the wet etching and the dry etching may be conducted (FIG. 2C).

Then, a conductive layer is formed so as to cover the contact holes and the first interlayer insulating layer 60. A connection portion 61a, a wiring 61b, or the like are formed by processing the conductive layer into a desired shape. This wiring may be a single layer of aluminum; copper; an alloy including aluminum, carbon and nickel; an alloy including aluminum, carbon and molybdenum; or the like. In this embodiment mode, the wiring is formed in a multilayer structure in which molybdenum, aluminum, molybdenum are stacked in this order. Alternatively, a structure in which titanium, aluminum, and titanium are stacked or a structure in which titanium, titanium nitride, aluminum, and titanium are stacked, is also applicable (FIG. 2D).

A second interlayer insulating layer 63 is formed so as to cover the connection portion 61a, the wiring 61b, and the first interlayer insulating layer 60. As the material of the second interlayer insulating layer 63, an applied film having a self-flattening property such as a film of acrylic, polyimide, siloxane, or the like is preferable. In this embodiment mode, the second interlayer insulating layer 63 is formed with siloxane (FIG. 2E).

Next, an insulating layer may be formed with silicon nitride or the like over the second interlayer insulating layer 63. This is done to prevent the second interlayer insulating layer 63 from being etched more than necessary in a later step of etching a pixel electrode. Therefore, the insulating layer is not necessary in particular when the difference of the etching rate is large between the pixel electrode and the second interlayer insulating layer. Next, a contact hole penetrating the second interlayer insulating layer 63 to reach the connection portion 61a is formed.

Next, after a light-transmitting conductive layer is formed to cover the contact hole and the second interlayer insulating layer 63 (or the insulating layer), the light-transmitting conductive layer is processed to form the first electrode 64 of the thin film light-emitting element. Here, the first electrode 64 electrically contacts the connection portion 61a.

As the material of the first electrode 64, it is preferable to use a conductive film of a metal such as aluminum (Al), silver (Ag), gold (Au), platinum (Pt), nickel (Ni), tungsten (W), chromium (Cr), molybdenum (Mo), iron (Fe), cobalt (Co), copper (Cu), palladium (Pd), lithium (Li), cesium (Cs), magnesium (Mg), calcium (Ca), strontium (Sr), or titanium (Ti); or an alloy including the metal described above; an nitride of a metal (such as TiN); a metal oxide such as ITO (indium tin oxide), ITO containing silicon (ITSO), IZO (indium zinc oxide) in which zinc oxide (ZnO) is mixed into indium oxide; can be used.

In addition, the electrode from which luminescence is extracted may be formed using a light-transmitting conductive film, and an ultrathin film of a metal such as Al or Ag is used, besides metal oxides such as ITO, ITSO, and IZO. In the case of extracting luminescence from the second electrode, a material that has a high reflectivity (such as Al or Ag) can be used for the first electrode. In this embodiment mode, ITSO is used for the first electrode 64 (FIG. 3A).

Next, an insulating film including an organic material or an inorganic material is formed to cover the second interlayer insulating layer 63 (or the insulating layer) and the first electrode 64. Subsequently, the insulating layer is processed so that a portion of the first electrode 64 is exposed, and a partition 65 is thus formed. As a material for the partition 65, a photosensitive organic material (such as acrylic and polyimide) is preferably used. However, a non-photosensitive organic material or inorganic material may be used for forming the partition 65. Further, as a material for the partition 65, a black pigment or dye such as carbon nitride may be dispersed with the use of a dispersant to make the partition 65 black as a black matrix. It is preferable that an end surface of the partition 65 on the side where it contacts with the first electrode 64 have a curvature and a tapered shape in which the curvature continuously changes (FIG. 3B).

Next, a hole injecting layer is formed to cover the first electrode 64, which is not covered with the partition 65. The hole injecting layer is formed using any one of hole injecting materials of the present invention which are represented by the general formulas (1) and (3), and the structural formulas (6) to (59) in Embodiment Mode 1, and an inkjet method may be employed for the application. Then, Alq₃ and coumarin 6 are deposited to have a thickness of 35 nm with the weight ration of 1:0.005 as the light-emitting layer, and Alq₃ is deposited to have a thickness of 10 nm as the electron transporting layer. Thus, an organic layer 66 including the hole injecting layer, the light-emitting layer, and the electron transporting layer is formed over the first electrode 64.

Subsequently, the second electrode 67 is formed to cover the organic layer 66. In this way, a light-emitting element 93 formed by sandwiching the organic layer 66 including the light-emitting layer between the first electrode 64 and the second electrode 67 can be manufactured, and luminescence can be obtained by applying a higher potential to the first electrode than to the second electrode. As a material for forming the second electrode 67, the same materials as that for the first electrode can be used. In this embodiment mode, aluminum is used for the second electrode.

In addition, the hole injecting layer is formed on the first electrode in this embodiment mode. However, an electron transporting layer may be provided on the first electrode to have a reversed stacked structure. In this case, luminescence can be obtained by making a voltage that is applied to the first electrode lower than a voltage that is applied to the second electrode.

After that, a silicon oxide film containing nitrogen is formed as a second passivation film by a plasma CVD method. In the case of using a silicon oxide film containing nitrogen, a silicon oxynitride film that is formed with SiH₄, N₂O, or NH₃, a silicon oxynitride film that is formed with SiH₄ and N₂O, or a silicon oxynitride film that is formed with a gas of SiH₄ and N₂O diluted with Ar may be formed by a plasma CVD method.

A silicon oxynitride hydride film formed with SiH₄, N₂O, or H₂ may be applied as the first passivation film. The structure of the first passivation film is not limited to a single layer structure. The first passivation film may have a single layer structure or stacked structure using another insulating layer including silicon. In addition, a multilayer film of a carbon nitride film and a silicon nitride film, a multilayer film of a styrene polymer, a silicon nitride film, or a diamond like carbon film may be formed instead of the silicon oxide film containing nitrogen.

Subsequently, sealing of a display portion is performed to protect the light-emitting element from substances, such as water, that accelerate deterioration. In the case of using an opposite substrate for sealing, the opposite substrate is attached with the use of an insulating sealing agent so that an external connecting portion is exposed. The space between the opposite substrate and the element substrate may be filled with an inert gas such as dried nitrogen, or the sealing agent may be applied to the whole surface of a pixel portion, and it is attached to the opposite substrate. It is preferable to use an ultraviolet curing resin or the like for the sealing agent. The sealing agent may be mixed with a drying agent or particles for keeping a gap between the substrates constant. Then, a light-emitting device is completed by attaching a flexible wiring substrate to the external connecting portion.

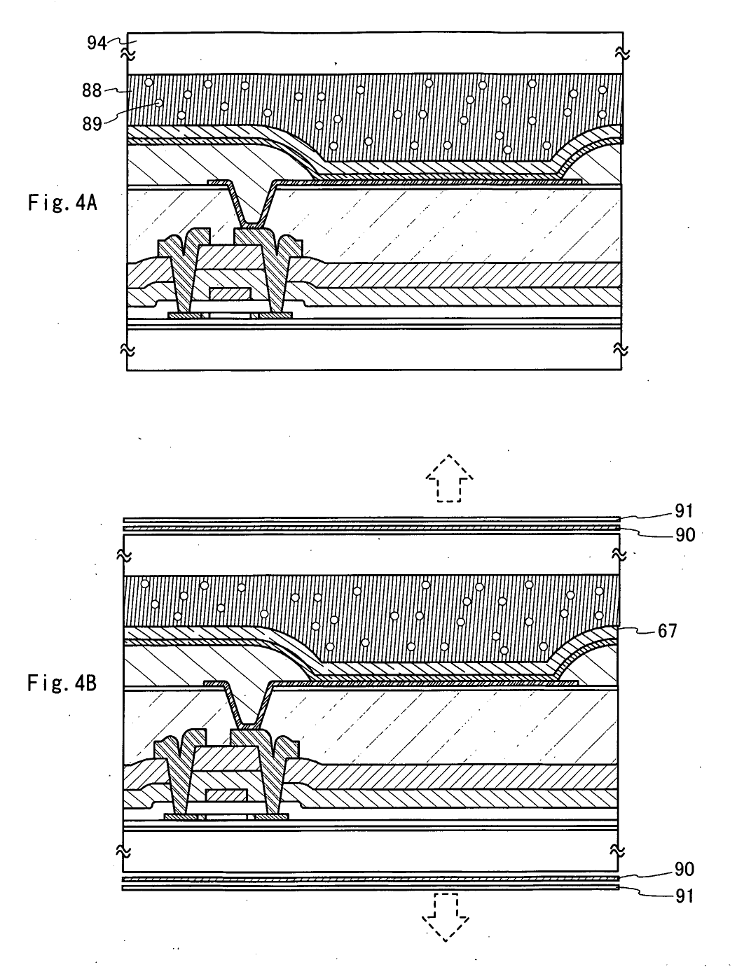

Examples of the structure of the thus manufactured display device will be described with reference to FIGS. 4A and 4B. It is to be noted that the same reference numeral is adopted for portions that carry out the same function even when the shapes of the portions are different from each other, and the description thereof can be omitted. In this embodiment mode, the thin film transistor 70 that has an LDD structure is connected to the light-emitting element 93 via the connecting potion 61a.

FIG. 4A shows a structure in which the first electrode 64 is formed with a light-transmitting conductive film, and light emitted from the organic layer 66 is extracted from the substrate 50 side. Further, reference numeral 94 denotes an opposite substrate, which is attached to the substrate 50 with the use of a sealing agent or the like after the light-emitting element 93 is formed. A light-transmitting resin 88 or the like is provided between the opposite substrate 94 and the element, and sealing is conducted, so that the light-emitting element 93 can be prevented from being deteriorated due to moisture. In addition, it is preferable that the resin 88 be hygroscopic. Further, it is more preferable to disperse a highly light-transmitting drying agent 89 in the resin 88, since the effect of moisture can be further suppressed.

FIG. 4B shows a structure in which both the first electrode 64 and the second electrode 67 are each formed of a light-transmitting conductive film, and light can be extracted through both the substrate 50 and the opposite substrate 94. Further, in this structure, the screen can be prevented from being seen through by providing polarization plates 90 on the outer sides of the substrate 50 and the opposite substrate 94, and the visibility is thus improved. On the outer sides of the polarization plates 90, protective films 91 are preferably provided.

It is to be noted that either an analog video signal or a digital video signal may be used for the display device of the present invention which has a display function. In the case of a digital video signal, there is a video signal made by voltage or one made by current. When a light-emitting element emits light, as a video signal to be input to the pixel, there are given a constant-voltage signal and a constant-current voltage. In a case of a constant voltage video signal, the voltage that is applied to the light-emitting element is constant or the current that flows in the light-emitting element is constant. Further, in a case of a constant-current video signal, the voltage that is applied to the light-emitting element is constant or the current that flows in the light-emitting element is constant. The case that the voltage that is applied to the light-emitting element is constant is referred to as a constant voltage driving, while the case that the current that flows in the light-emitting element is constant is referred to as constant current driving. In the constant-current driving, a constant current flows regardless of change in the resistance of the light-emitting element. Either the driving method with voltage or the driving method with current may be adopted for a light-emitting display device and the driving method according to the present invention. Either the constant-voltage driving or the constant-current driving may be used.

This embodiment mode can be used freely combined with an appropriate structure described in Embodiment Modes 1 to 3.

### Embodiment Mode 5

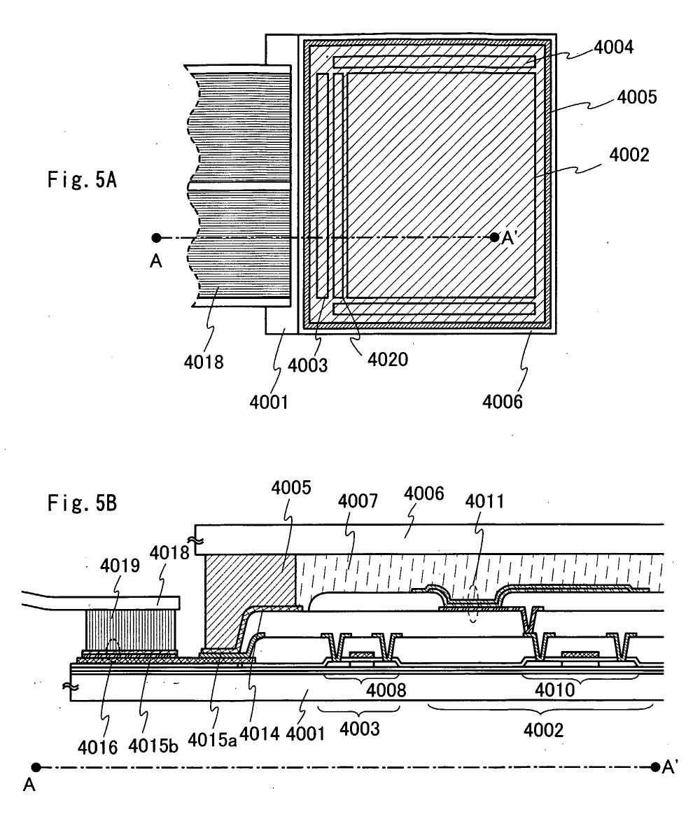

In Embodiment Mode 5, the appearance of a panel of a light-emitting device that corresponds to one mode of the present invention will be described with reference to FIGS. 5A and 5B. FIG. 5A is a top view of a panel in which transistors and a light-emitting element that are formed over a substrate are sealed with a sealing agent between the substrate and an opposite substrate 4006. FIG. 5B corresponds to a cross-sectional view of FIG. 5A. The structure of the light-emitting element in the panel is the structure as described in Embodiment Mode 4.

The sealing agent 4005 is provided so as to surround a pixel portion 4002, a signal line driver circuit 4003, and a scan line driver circuit 4004 that are provided over the substrate 4001. Further, the opposite substrate 4006 is provided over the pixel portion 4002, the signal line driver circuit 4003, and the scan line driver circuit 4004. Accordingly, the pixel portion 4002, the signal line driver circuit 4003, and the scan line driver circuit 4004 are sealed, together with a filling agent 4007, by the substrate 4001, the sealing agent 4005, and the opposing substrate 4006.

Further, each of the pixel portion 4002, the signal line driver circuit 4003, and the scan line driver circuit 4004 over the substrate 4001 has a plurality of thin film transistors. In FIG. 5B, the thin film transistor 4008 included in the signal line driver circuit 4003 and the thin film transistor 4010 included in the pixel portion 4002 are shown.

Further, the light-emitting element 4011 is electrically connected to the thin film transistor 4010.

Further, a leading wiring 4014 corresponds to a wiring for supplying signals or a power supply voltage to the pixel portion 4002, the signal driver circuit 4003, and the scan line driver circuit 4004. The lead wiring 4014 is connected to a connecting terminal 4016 via leading wirings 4015a and 4015b. The connecting terminal 4016 is electrically connected to a terminal of a flexible printed circuit (FPC) 4018 via an anisotropic conductive film 4019.

As the filling agent 4007, ultraviolet curing resins and thermal curing resin can be used besides an inert gas such as nitrogen or argon, and polyvinylchloride, acrylic, polyimide, epoxy resin, silicon resin, polyvinyl butylal, or ethylene vinylene acetate can be used.

It is to be noted that a panel in which a pixel portion that has a light-emitting element is formed and a module in which an IC is mounted on the panel are included in the category of the light-emitting device of the present invention.

Panels and modules that have the structure described in this embodiment mode are heat-resistant and durable panels and modules, since a composite material having a skeleton made by a siloxane bond is used for light-emitting elements. Further, since a composite material further added with a material that can transfer electrons with an organic group having an electron injecting or transporting property or a hole injecting or transporting property is used for the skeleton, panels and modules that have an improved electron injecting or transporting property or an improved hole injecting or transporting property and further has improved conductivity can be provided.

Further, when the functional layer on the first electrode is formed to have a thickness of 100 nm or more with the use of the composite material that has an improved electron injecting or transporting property or an improved hole injecting or transporting property and further has improved conductivity, occurrence of defects due to dust and the like on the first electrode can be reduced without causing a significant increase in the driving voltage.

This embodiment mode can be freely combined with an appropriate structure described in Embodiment Modes 1 to 4.

### Embodiment Mode 6

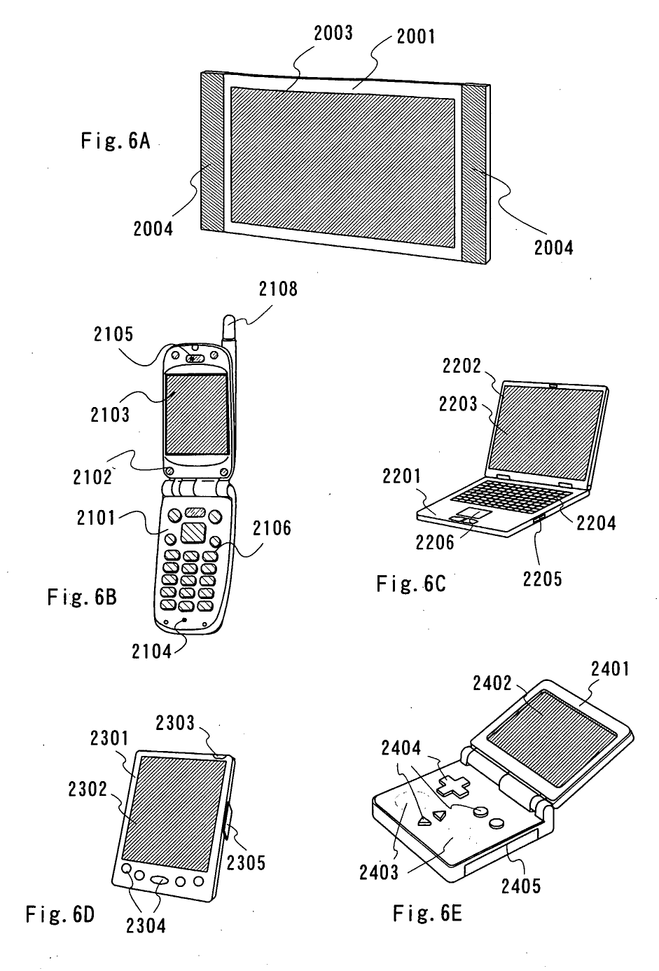

Electronic devices according to the present invention, which are each mounted with a module like the example shown in Embodiment Mode 5, include a camera such as a video camera or a digital camera; a goggle-type display (head mount display); a navigation system; a sound reproduction device (a car audio component or the like); a computer; a game machine; a portable information terminal (a mobile computer, a cellular phone, a portable game machine, an electronic book, or the like); and an image reproduction device equipped with a recording medium (specifically, a device equipped with a display, which can reproduce content of a recording medium such as a Digital Versatile Disc (DVD) and display the image). Specific examples of these electronic devices are shown in FIGS. 6A to 6E.

FIG. 6A is a light-emitting display device, such as a television set or a monitor of a personal computer. The light-emitting display device includes a frame body 2001, a display portion 2003, a speaker portion 2004, and the like. The light-emitting display device of the present invention is a light-emitting display device which can be manufactured at a low cost, since the material for a light-emitting element is used for the light-emitting element of the display portion 2003. A pixel portion is preferably provided with a polarization plate or a circularly polarization plate in order to enhance the contrast. For example, it is preferable to provide films in the order corresponding to a quarter-wavelength plate, a half-wavelength plate, and a polarization plate over a sealing substrate. Further, an anti-reflective film may be provided over the polarization plate.

FIG. 6B is a cellular phone, which includes a main body 2101, a frame body 2102, a display portion 2103, a sound input portion 2104, a sound output portion 2105, an operation key 2106, an antenna 2108, and the like. The cellular phone of the present invention is a cellular phone which can be manufactured at a low cost, since the material for a light-emitting element described in Embodiment Mode 1 is used for the light-emitting element of the display portion 2103.

FIG. 6C is a computer, which includes a main body 2201, a frame body 2202, a display portion 2203, a keyboard 2204, an external connection port 2205, a pointing mouse 2206, and the like. The computer of the present invention is a computer which can be manufactured at a low cost, since the material for a light-emitting element described in Embodiment Mode 1 is used for the light-emitting element of the display portion 2203. In FIG. 6C, a laptop computer is shown as an example. However, the present invention can be applied to a desktop computer or the like.

FIG. 6D is a mobile computer, which includes a main body 2301, a display portion 2302, a switch 2303, an operation key 2304, an infrared port 2305, and the like. The mobile computer of the present invention is a mobile computer which can be manufactured at a low cost, since the material for a light-emitting element described in Embodiment Mode 1 is used for the light-emitting element of the display portion 2302.

FIG. 6E is a portable game machine, which includes a frame body 2401, a display portion 2402, a speaker portion 2403, operation keys 2404, a recording medium insert portion 2405, and the like. The portable game machine of the present invention is a portable game machine which can be manufactured at a low cost, since the material for a light-emitting element described in Embodiment Mode 1 is used for the light-emitting element of the display portion 2402.

As described above, the application range of the present invention is extremely wide, and the present invention can be thus applied for electronic devices in all fields.

### Embodiment Mode 7

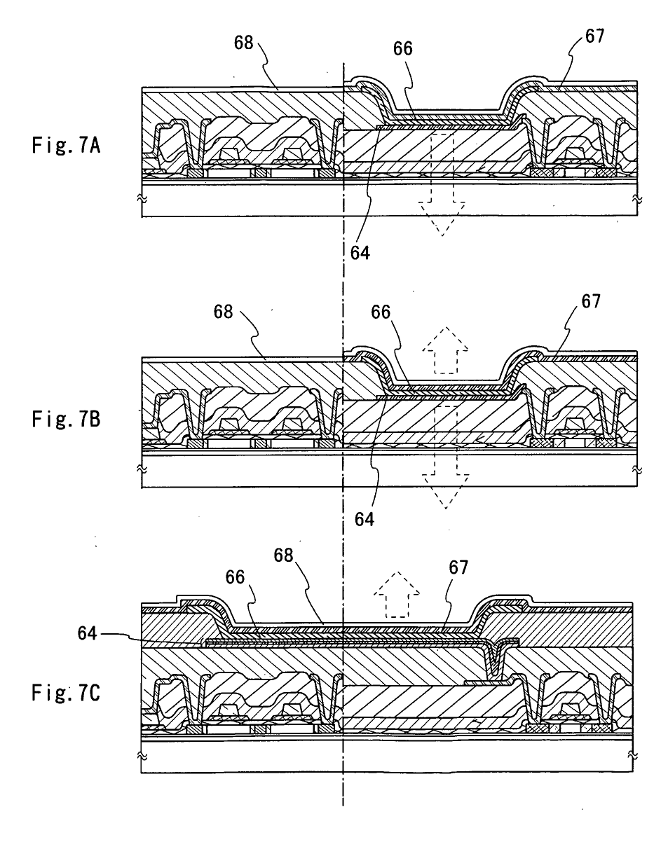

FIGS. 7A to 7C respectively show examples of bottom-emission, dual-emission, and top-emission devices. Each of FIGS. 7A and 7B shows a structure for the case of forming a first interlayer insulating layer in FIG. 7C with the use of a self-flatness material and forming a wiring that is connected to a thin film transistor and a first electrode 64 of a light-emitting element over the same interlayer insulating layer. In FIG. 7A, only the first electrode 64 for the light-emitting element is formed using a light-transmitting material to provide a bottom-emission structure in which light is emitted toward the bottom of the light-emitting device. In the case of FIG. 7B, a dual-emission light-emitting display device that is able to extract light from the both sides as shown in FIG. 7B can be obtained by using a light-transmitting material such as ITO, ITSO, or IZO also for a second electrode 67. It is to be noted that a material such as aluminum or silver, which is not light-transmitting in a thick film, gets to have a light-transmitting property when the thickness is made thinner. Therefore, also when the second electrode 67 is formed by using a film of aluminum or silver that is thin enough to have a light-transmitting property, a dual-emission device can be obtained.

### Embodiment Mode 8

In Embodiment Mode 8, a pixel circuit and a protection circuit that are included in the panel or module described in Embodiment Mode 5 and operations thereof will be described. It is to be noted that the cross-sectional views shown in FIGS. 2A to 2E and FIGS. 3A to 3C correspond to cross-sectional views of a driving TFT 1403 and a light-emitting element 1405.

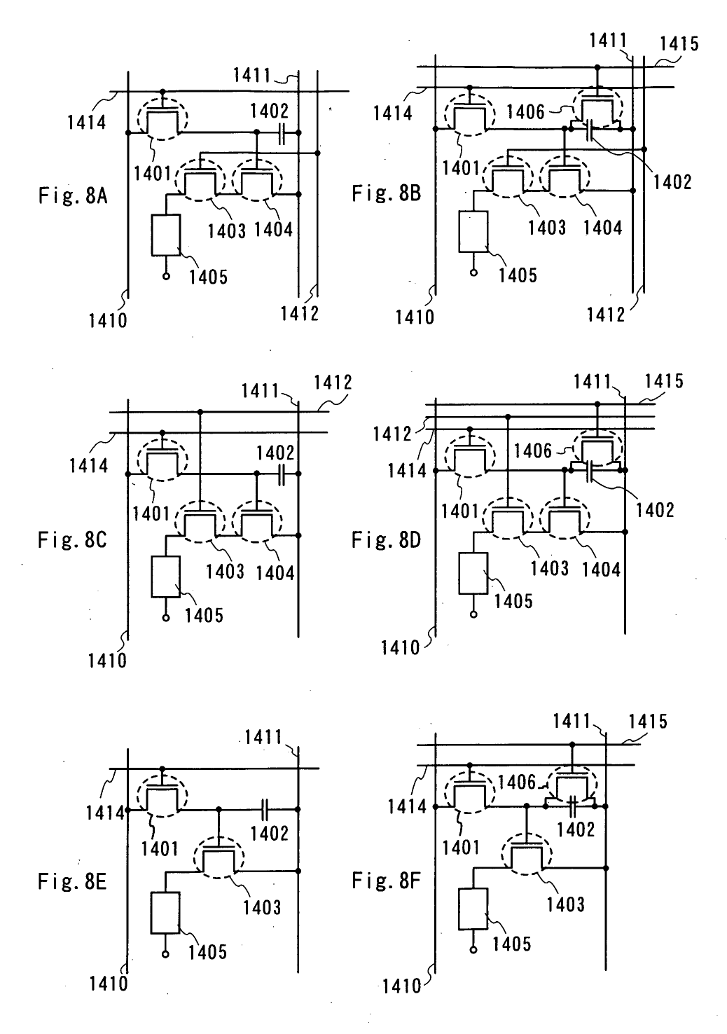

In the pixel shown in FIG. 8A, a signal line 1410, power supply lines 1411 and 1412 are arranged in a column direction, and a scan line 1414 is arranged in a row direction. The pixel also includes a switching TFT 1401, a driving TFT 1403, a current controlling TFT 1404, a capacitor 1402 and a light-emitting element 1405.

The pixel shown in FIG. 8C, which basically has the same structure as the pixel shown in FIG. 8A, is different only in that a gate electrode of a TFT 1403 is connected to a power supply line 1412 arranged in a row direction. Namely, each of FIGS. 8A and 8C illustrates the same equivalent circuit diagram. However, when the case of arranging the power supply line 1412 in the column direction (FIG. 8A) is compared to the case of arranging the power supply line 1412 in the row direction (FIG. 8C), the power supply lines are each formed with a conductive film in a different layer. In this embodiment mode, attention is given to the wiring connected to the gate electrode of each driving TFT 1403, and FIGS. 8A and 8C are separately illustrated to indicate that the layers for forming these wirings are different from each other.

In each of the pixels shown in FIGS. 8A and 8C, the driving TFT 1403 and the current controlling 1404 are connected in series in the pixels. It is preferable that the channel length L (1403) and channel width W (1403) of the driving TFT 1403 and the channel width L (1404) and channel width W (1404) of the current controlling TFT 1404 satisfy L (1403)/W (1403):L (1404)/W (1404)=5 to 6000:1.

It is to be noted that the driving TFT 1403 operates in the saturation region and functions to control a current value that is applied to the light-emitting element 1405, while the current controlling TFT 1404 operates in the linear region and functions to control current supply to the light-emitting element 1405. Both of the TFTs preferably have the same conductivity type in the manufacturing process, and are formed as N-channel TFTs in this embodiment mode. For the driving TFT 1403, not only an enhancement mode TFT but also a depletion mode TFT may be used. In the present invention having the structure described above, the current controlling TFT 1404 operates in the linear region. Therefore, slight fluctuation in Vgs of the current controlling TFT 1404 has no influence on a current value that is applied to the light-emitting element 1405. Namely, the current value that is applied to the light-emitting element 1405 can be determined by the driving TFT 1403, which operates in the saturation region. The structure described above makes it possible to improve luminance unevenness of light-emitting elements due to variations in characteristics of TFTs and to provide a display device with enhanced image quality.

In each of the pixels shown in FIGS. 8A to 8D, the switching TFT 1401 controls input of a video signal to the pixel. When the switching TFT 1401 is turned ON, a video signal is input to the pixel. Then, the voltage of the video signal is held in the capacitor 1402. Although each of FIGS. 8A and 8C illustrates the structure in which the capacitor 1402 is provided, the present invention is not limited to this structure. The capacitor 1402 may be omitted when a gate capacitance or the like can cover the capacitance for holding a video signal.

The pixel shown in FIG. 8B, which basically has the same pixel structure as FIG. 8A, is different only in that a TFT 1406 and a scan line 1415 are additionally provided. Similarly, the pixel shown in FIG. 8D, which basically has the same pixel structure as FIG. 8C, is different only in that the TFT 1406 and a scan line 1415 are additionally provided.

The switching (ON/OFF) of the TFT 1406 is controlled by the scan line 1415 provided additionally. When the TFT 1406 is turned ON, a charge held in the capacitor 1402 is discharged to turn OFF the current controlling TFT 1404. Namely, the arrangement of the TFT 1406 makes it possible to bring the light-emitting element 1405 forcibly into a state where no current flows thereto. Therefore, the TFT 1406 can be referred to as an erasing TFT. Thus, in the structures shown in FIGS. 8B and 8D, an emission period can be started simultaneously with or immediately after a writing period without waiting for completion of writing signals to all pixels, and thus, the duty ratio can be improved.

In the pixel shown in FIG. 8E, a signal line 1410 and a power supply line 1411 are arranged in a column direction, and a scan line 1414 is arranged in a row direction. The pixel also includes a switching TFT 1401, a driving TFT 1403, a capacitor 1402 and a light-emitting element 1405. The pixel shown in FIG. 8F, which basically has the same pixel structure as FIG. 8E, is different only in that a TFT 1406 and a scan line 1415 are additionally provided. Also in the structure shown in FIG. 8F, the arrangement of the TFT 1406 makes it possible to improve the duty ratio.

As described above, various pixel circuits can be employed. In particular, in the case of forming a thin film transistor with an amorphous semiconductor film, it is preferable to make a semiconductor film for the driving TFT 1403 larger. Therefore, for the pixel circuit described above, it is preferable to employ a top emission type in which light from an electroluminescent element is emitted through a sealing substrate.

This active-matrix light-emitting device can be driven at a low voltage, and is thus considered to be advantageous, when the pixel density is increased since a TFT is provided in each pixel.

In this embodiment mode, an active-matrix light-emitting device in which a TFT is provided in each pixel is described. However, a passive-matrix light-emitting device in which a TFT is provided for each column can be also formed. The passive-matrix light-emitting device has a high aperture ratio, since a TFT is not provided for each pixel. In the case of a light-emitting device in which luminescence is emitted through the both sides of a light-emitting element, the transmittance is increased when a passive-matrix light-emitting device is used.

A display device according to the present invention, which further includes a pixel circuit like these, can be a light-emitting device that has each of the features described above, since a material that is suitable for the structure and required performance of a light-emitting element can be used for an electrode of the light-emitting element included in the display device.

Subsequently, a case of providing diodes as protection circuits for a scan line and a signal line will be described with reference to the equivalent circuit shown in FIG. 8E.

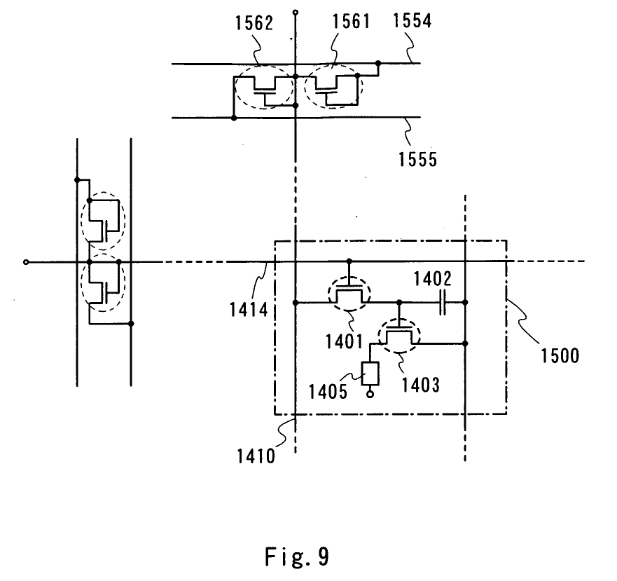

In FIG. 9, switching TFTs 1401 and 1403, a capacitor 1402, and a light-emitting element 1405 are provided in a pixel portion 1500. For a signal line 1410, diodes 1561 and 1562 are provided. The diodes 1561 and 1562 are manufactured in accordance with the embodiment modes described above in the same way as the switching TFTs 1401 and 1403, and each of the diodes 1561 and 1562 includes a gate electrode, a semiconductor layer, a source electrode, and a drain electrode, and the like. Each of the diodes 1561 and 1562 operates as a diode by connecting the gate electrode to the drain electrode or the source electrode.

Common potential lines 1554 and 1555 that are connected to the diodes are formed in the same layer as the gate electrode. Accordingly, it is necessary to form contact holes in a gate insulating layer so that they are each connected to the source electrode or drain electrode of the diodes.

Diodes that are provided for a scan line 1414 have the same structure.

As described above, a protective diode that is provided for an input state can be formed at the same time according to the present invention. It is to be noted that the position in which the protection diode is formed is not limited to this, and the protection circuit can be provided between a driver circuit and a pixel.

A display device of the present invention, which has this protection circuit, can be manufactured since the material for a light-emitting element described in Embodiment Mode 1 is used for the light-emitting element of the display device. Moreover, the reliability of the display device can be further enhanced by including the structure described above.

### Example 1

### Synthesis Example

As a synthesis example, a synthesis method of poly{4-[N-(4-diphenylamino phenyl)-N-phenyl]aminostyrene} (PStDPA) will be described.

[Step 1] A synthesis of N-(4-diphenylamino)phenylaniline is described.

In a 1000 mL erlenmayer flask, 25.19 g (0.102 mol) of triphenylamine, 18.05 g (0.102 mol) of N-bromosuccinimide, and 400 ml of ethyl acetate were put, and stirred at room temperature in the air overnight (for about 12 hours). After completion of the reaction, the organic layer was washed twice with a saturated aqueous solution of sodium carbonate, then, the water layer was extracted twice with ethyl acetate, and the ethyl acetate layer and the organic layer were washed with a saturated aqueous solution of sodium chloride. After drying with magnesium sulfate, natural filtration, and concentration, the obtained colorless solid was recrystallized with ethyl acetate and hexane to obtain of 22.01 g of a colorless powdery solid with the yield of 66%. Nuclear magnetic resonance (NMR) was used to confirm that this colorless powdery solid was N,N-diphenyl-N-(4-bromophenyl)amine. The measurement result by nuclear magnetic resonance (NMR) is shown below.

¹H-NMR data of this compound is shown below.

¹H-NMR (300 MHz, CDCl₃) δ ppm: 7.32 (d, 2H, J=8.7 Hz), 7.29-7.23 (m, 4H), 7.08-7.00 (m, 6H), 6.94 (d, 2H, J=8.7 Hz).

In addition, a synthesis scheme of N,N-diphenyl-N-(4-bromophenyl)amine is shown by the following formula.

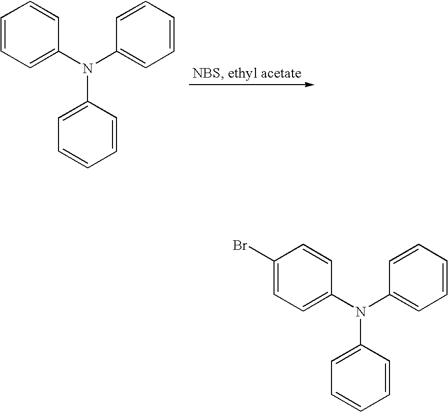

Dehydrogenated toluene solution (5 mL) including N,N-diphenyl-N-(4-bromophenyl)amine (559 mg, 6 mmol), dibenzylideneacetone palladium (Pd(dba)₂) (345 mg, 0.6 mmol), sodium-tert-butoxide (t-BuONa) (577 mg, 6 mmol) was deaerated. Thereafter, aniline (559 mg, 6 mmol) and tri-tert-butylphosphine (P(t-Bu)₃) (0.37 mL, 1.8 mmol) were added thereto, and stirred under nitrogen atmosphere at 80° C. for 5 hours. It was confirmed using a thin film chromatography that N,N-diphenyl-N-(4-bromophenyl)amine as a raw material was lost. After that, saturation saline was added thereto, and a water layer was extracted with about 100 ml of ethyl acetate. The organic layer was dehydrogenated with magnesium sulfate and filtered. The filtrate was concentrated, and then refined in a silica gel column of ethyl acetate:hexane=1:20 (the yield: 42%).

It was confirmed with a nuclear magnetic resonance method (NMR) that the obtained solid matter was N-(4-diphenylamino)phenylaniline.

¹H-NMR of this compound is shown as follows. ¹H-NMR (300 MHz, CDCl₃) dppm: 7.32 (d, 2H, J=8.7 Hz), 7.29-7.23 (m, 5H), 7.08-7.00 (m, 6H), 6.94 (d, 2H, J=8.7 Hz)

A synthesis scheme of N-(4-diphenylamino)phenylaniline is shown by the following formula.

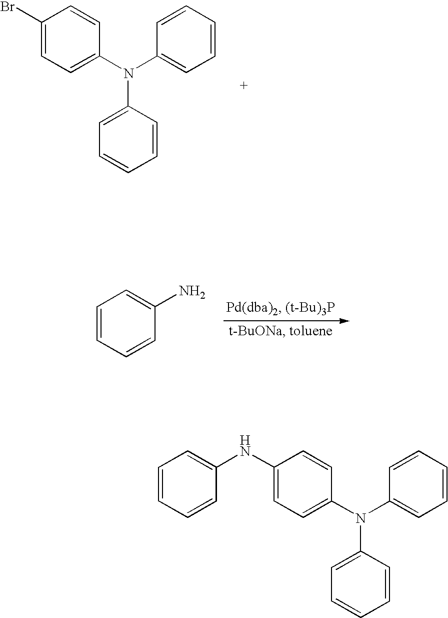

### [Step 2]

A synthesis of a compound of 4-[N-(4-diphenylaminophenyl)-N-phenyl]aminobenzaldehyde will be described.

Under nitrogen, 4.09 g (17.9 mmol) of 2-(4-bromophenyl)-1,3-dioxolane, and 0.3 mL of P(t-Bu)₃ 43% hexane solution were added to 100 mL of a dried toluene suspension including 10.0 g (29.8 mmol) of the synthesized N-(4-diphenylamino)phenylaniline, 2,200 mg (0.348 mmol) of Pd(dba), 11.0 g (114 mmol) of t-BuONa, and stirred for 3 hours at 80° C.

The reaction mixture was filtered through florisil, alumina and Celite®. The obtained filtrate was washed with a saturated aqueous solution. This organic layer was dried with magnesium sulfate, filtered and concentrated. The obtained yellow solid matter was dissolved in about 100 mL of tetrahydrofuran (THF), and then, about 50 mL of 3% hydrochloric acid was added to the solution and stirred at a room temperature overnight (for about 12 hours). The reaction solution was extracted with ethyl acetate, and then, the obtained oily crude product was refined in column chromatography (the developing solvent was a mixture solvent of hexane and ethyl acetate) to obtain a yellow solid matter of 6.76 g with the yield of 86%. It was confirmed with a nuclear magnetic resonance method (NMR) that the obtained yellow solid matter was 4-[N-(4-diphenylaminophenyl)-N-phenyl]aminobenzaldehyde.

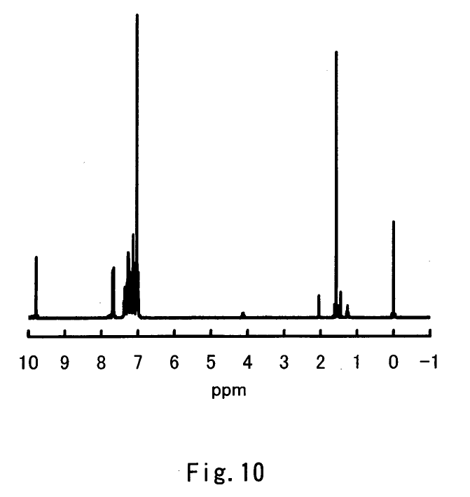

FIG. 10 shows an NMR spectrum of this compound. In addition, ¹H-NMR is as follows: ¹H-NMR (300 MHz, CDCl₃) δ ppm: 9.79 (s, 1H), 7.68 (d, J=9.0 Hz), 6.89-7.40 (m, 23H).

In addition, a synthesis scheme of 4-[N-(4-diphenylaminophenyl)-N-phenyl]aminobenzaldehyde is shown by the next formula.

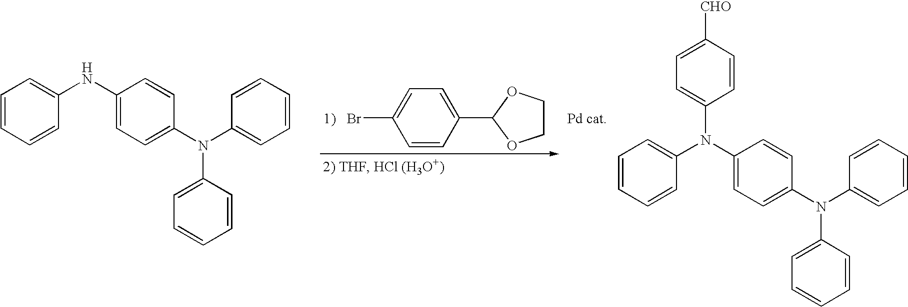

[Step 3] A synthesis of 4-[N-(4-diphenylaminophenyl)-N-phenyl]aminostyrene which is an organic compound and is one of monomers according to the present invention, will be described.

Under nitrogen, 15 mL of n-butyllithium (1.58 mol/L) was dropped, at −40° C., into a suspension of 9.31 g (23.0 mmol) of methyl triphenylphosphonium iodine suspended in dried tetrahydrofuran (100 mL). After the dropping, it was stirred at 0° C. for 1 hour. Into the obtained reaction solution, a dried tetrahydrofuran solution including 6.76 g (15.4 mmol) of 4-[N-(4-diphenylaminophenyl)-N-phenyl]aminobenzaldehyde synthesized in Step 2 was added, and stirred at a room temperature overnight (for about 12 hours).

After the reacted mixture was filtered through Celite®, water was added thereto and an organic layer was extracted with ethyl acetate. The organic layer was dried with magnesium sulfate, filtered, and concentrated, the obtained residue was refined in silica gel column chromatography (the developing solvent is a mixture solvent of hexane and ethyl acetate) to obtain a light-yellow solid matter of 4.54 g with the yield of 81%. It was confirmed with a nuclear magnetic resonance method (NMR) that the obtained light-yellow solid matter was 4-[N-(4-diphenylaminophenyl)-N-phenyl]aminostyrene (StDPA).

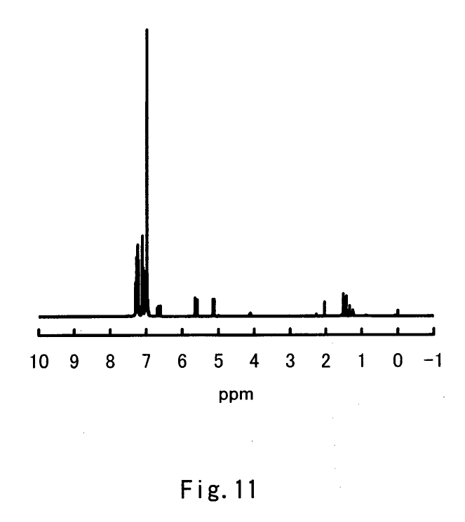

FIG. 11 shows an NMR spectrum of this compound. In addition, ¹H-NMR is as follows: ¹H-NMR (300 MHz, CDCl₃) δ ppm: 6.94-7.30 (m, 23H), 6.64 (dd, 1H, J=11, 18 Hz), 5.62 (dd, J=18 Hz), 5.13 (s, 1H, J=11 Hz).

In addition, a synthesis scheme of 4-[N-(4-diphenylaminophenyl)-N-phenyl]aminostyrene (StDPA) which is an organic compound and is one of monomers according to the present invention, is shown by the following formula.

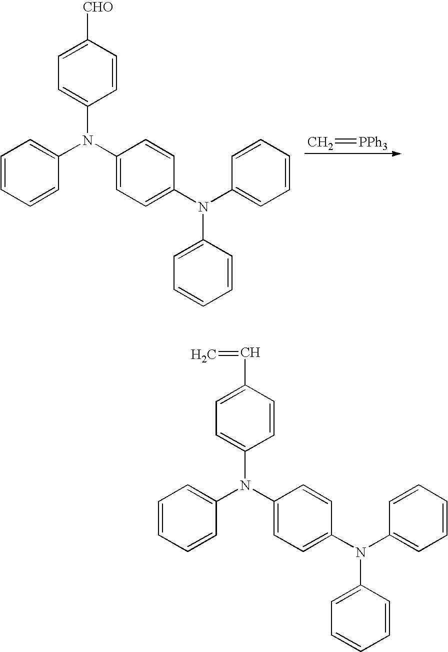

[Step 4] A synthesis of poly{4-[N-(4-diphenylaminophenyl)-N-phenyl]aminostyrene} which a hole-injecting material and is one of polymers according to the present invention, will be described.

A dried toluene mixture (10 mL) including 4.22 g (9.63 mmol) of 4-[N-(4-diphenylaminophenyl)-N-phenyl]aminostyrene (StDPA) synthesized in Step 3 and 112 mg (0.68 mmol) of Azoisobutyronitril was deaerated. After that, it was heated at 60° C. for 3 days. The reaction solution was precipitated again in ether, and the precipitated light-yellow solid matter was collected by filtrating. This light-yellow solid matter was dried under a reduced pressure to obtain 3.41 g of a light-yellow solid matter with the yield of 81%. It was confirmed with a nuclear magnetic resonance method (NMR) that the obtained yellow solid matter was poly{4-[N-(4-diphenylaminophenyl)-N-phenyl]aminostyrene} (PStDPA) which is a hole injecting material of one of polymers according to the present invention.

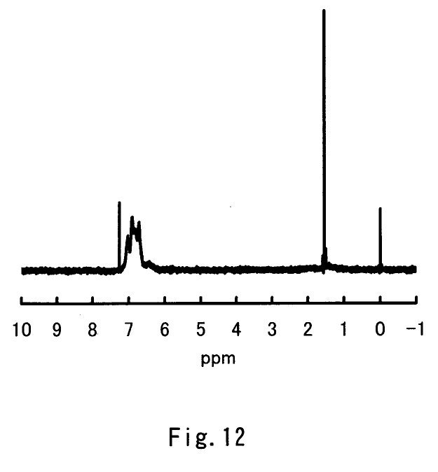

FIG. 12 shows a ¹H-NMR spectrum of this compound. In addition, ¹H-NMR is as follows: ¹H-NMR (300 MHz, CDCl₃) δ ppm: 1.20-2.5 (br, 3H), 6.30-7.40 (br, 23H).

A synthesis scheme of poly{4-[N-(4-diphenylaminophenyl)-N-phenyl]aminostyrene} (PStDPA) which is a hole injecting material of one of polymers according to the present invention, is shown by the following formula.

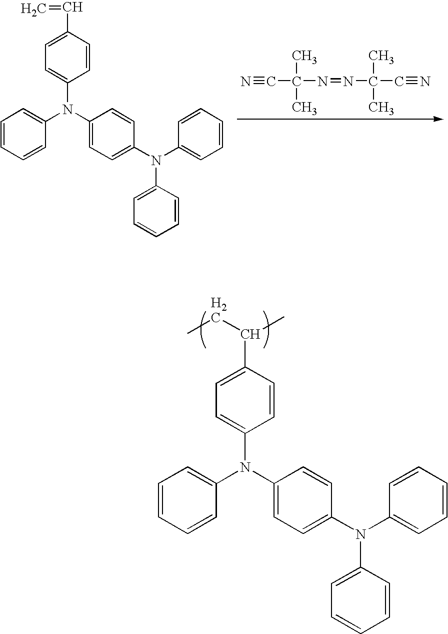

In this manner, PStDPA which is a hole injecting material of the present invention and a linear polymer having a repeating unit represented by the following formula, can be synthesized.

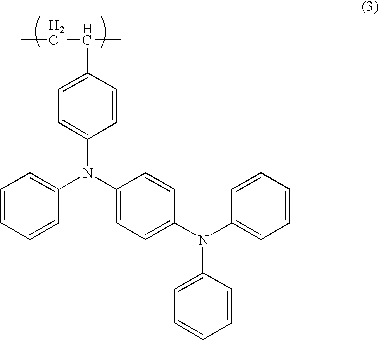

The molecular weight of the obtained PStDPA was measured using Gel Permeation Chromatography (GPC). It was known that the number average molecular weight was 18000, and the weight average molecular weight was 44000 in polystyrene conversion.

Then, the thermo physical property of the synthesized PStDPA is described.

The decomposition temperature of the synthesized PStDPA was measured at a rate of temperature rise 10° C./min, under nitrogen using Thermogravimetry-Differential Thermal Analysis (TG/DTA320 manufactured by Seiko Instruments). As a result, the weight loss starting temperature was 391° C.

In addition, the glass transition temperature of the synthesized PStDPA was examined with a differential scanning calorimetry (Pyris 1 DSC manufactured by Perkin Elmer Co., Ltd.) According to the measurement results, it was found that the glass transition temperature of the obtained PStDPA was 143° C.

As for an end group, a material of the present invention formed by the above synthesis method is a material having independently a group selected from the formulas (60) to (62) in the opposite ends, or a mixture including, in one of the opposite ends, a group selected from the formulas (60) to (62) and in the other end, a group selected from the formulas (63) to (66).

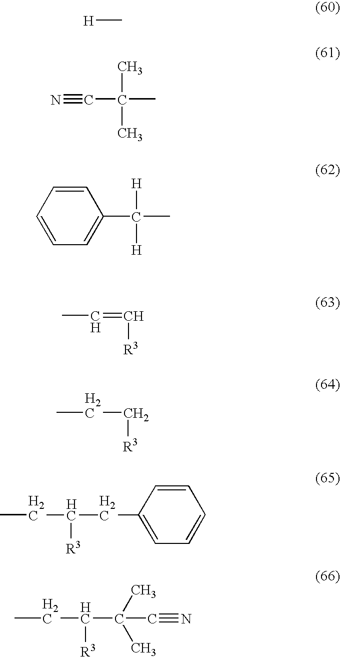

In the above formula, R³ represents a substituent represented below.

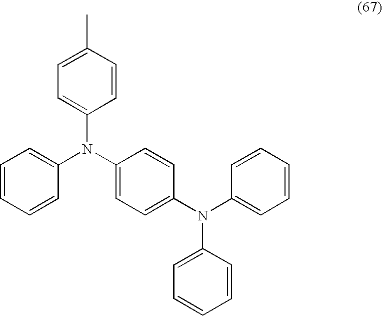

### Example 2

Example 2 shows measurement results of ionization potential and absorption spectrum of the PStDPA in a thin film state, which was synthesized in Example 1.

The thin film of PStDPA was formed over a glass substrate by a spin coating method.

A fabrication method of the synthesized PStDPA thin film is described in detail. 150 mg of PStDPA was dissolved in 30 ml of toluene. This solution was filtered through a 5 μm filter, and the filtrate was applied to coat the glass substrate. The application by the spin coating method was conducted as follows: rotation at 500 rpm was conducted for 2 seconds and then, rotation at 600 rpm was conducted for 60 seconds. Then, the glass substrate coated with PStDPA was baked at 120° C. for 1 hour, and the PStDPA thin film was completed.

The thickness of the obtained PStDPA thin film was measured with an ellipsometer (PZ2000 manufactured by Royal Philips Electronics N.V. in Japan), and the thickness was 30 nm.

The ionization potential and absorption spectrum of the synthesized PStDPA thin film were measured using a photoelectron spectrometer (manufactured by Riken Keiki Co., Ltd., AC-2) and an ultraviolet-visible spectrophotometer (manufactured by JASCO Corporation, V-550).

The ionization potential of the synthesized PDtDPA thin film was −5.19 eV from the measurement result.

In addition, the wavelength of the absorption edge on the side of the long wavelength of the absorption spectrum was made as an energy gap to obtain the LUMO level. The LUMO level was −1.89 eV.

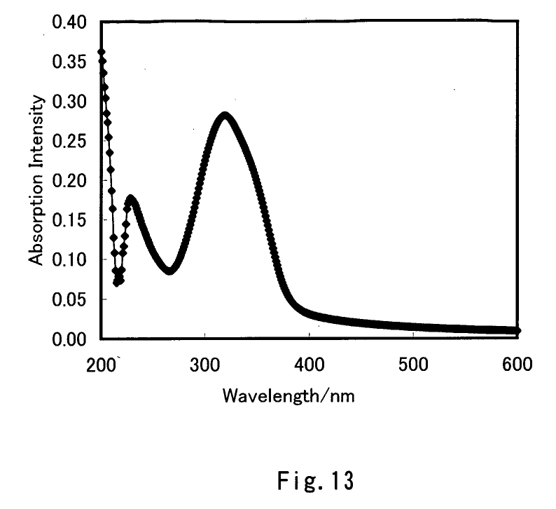

Note that FIG. 13 shows the measurement date.

### Example 3

Example 3 will describe a current-voltage characteristic of an element in which the PStDPA thin film was interposed between a pair of electrodes.

The structure of the element used for the measurement is described. The element was formed over a glass substrate, a first electrode (a transparent electrode:ITSO, 110 nm), a PStDPA thin film (30 nm), and a second electrode (aluminum, 150 nm) were stacked over the glass substrate.

Then, a manufacturing method of the element is described in detail.

The first electrode was formed over the glass substrate first. In this example, the first electrode was formed with ITSO by a sputtering method. The shape of the first electrode was 2 mm×2 mm in the present invention. Next, as a pretreatment for forming the PStDPA thin film over the first electrode, the surface of the substrate was washed with a porous resin (typically, one made of PVA (polyvinyl alcohol), nylon, or the like), and heated at 200° C. for 1 hour.

After the pretreatment, a solution in which 150 mg of PStDPA was dissolved in 30 ml of toluene was filtered through a 5 μm filter, and the filtrate was applied to coat the first electrode. The application by a spin coating was conducted as follows: rotation at 500 rpm was conducted for 2 seconds and then, rotation at 600 rpm was conducted for 60 seconds. The glass substrate coated with the PStDPA was baked at 120° C. for 1 hour, and the PStDPA thin film was completed.

150 nm of aluminum was deposited over the PStDPA thin film as the second electrode by a vacuum evaporation method to complete the element.

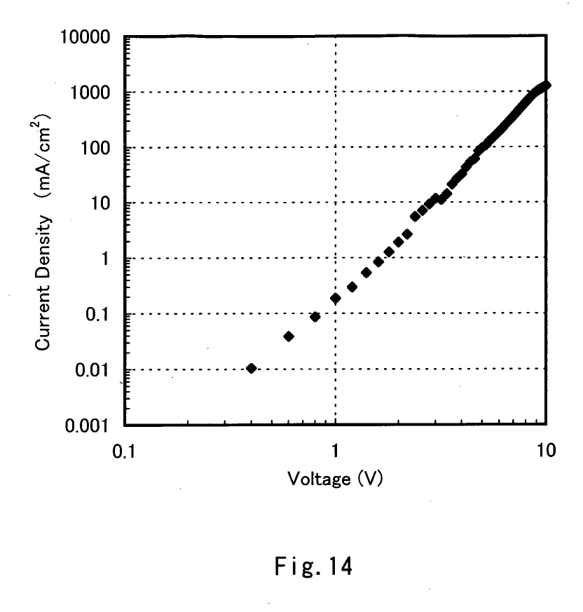

The current-voltage characteristic of the manufactured element by the above method was measured. FIG. 14 shows the measurement result. In FIG. 14, the horizontal axis shows voltage (a unit: V) and the vertical axis shows current density (a unit: mA/cm²).

The measurement was conducted using a 2400 type Source Meter manufactured by Keithley Instruments Inc, by applying voltage while using the first electrode of the element manufactured above as an anode, and the second electrode as a cathode. The voltage was applied at 0.2 V intervals in the range of 0V to 20 V, a current value of each voltage value was measured and converted into a current density.

According to the measurement result, as for the element having only the PStDPA thin film between the electrodes, it was known that the current density of 11.8 mA/cm² was obtained when a 3-V voltage was applied in the thickness of 30 nm. Further, since a region of ohm current was not shown, it was understood that a hole injection barrier into the PStDPA thin film from the first electrode was small and the carrier injecting property was superior.

### Example 4

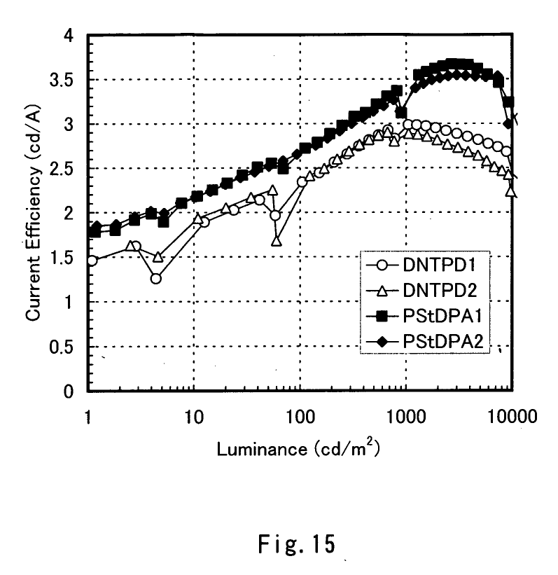

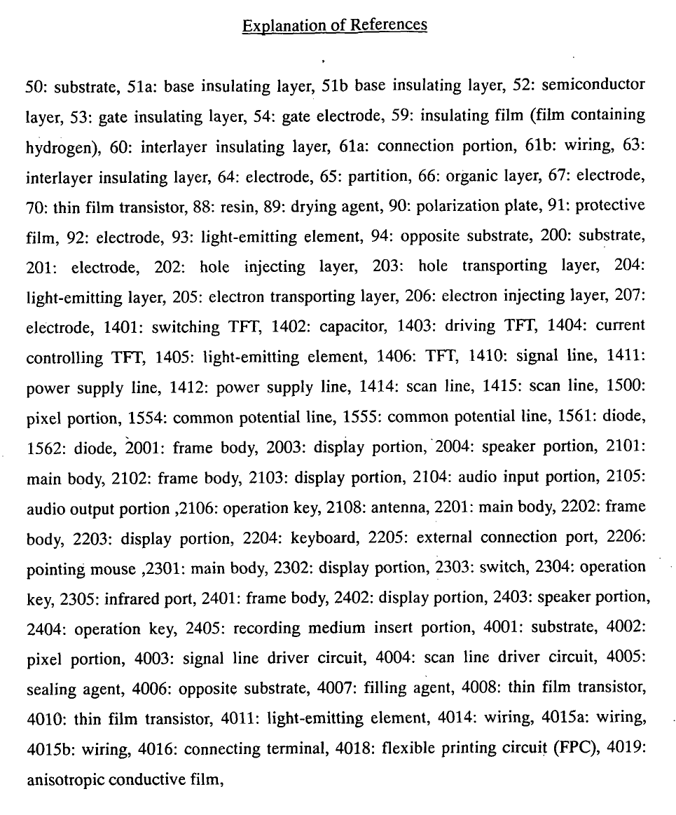

In Example 4, FIG. 15 shows a luminescence property of a light-emitting element using PStDPA as a hole injecting layer. In FIG. 15, the horizontal axis shows a luminance (cd/m²) and the vertical axis shows a current efficiency (cd/A).

The light-emitting element of this example which was used for measuring a luminescence property was formed over a glass substrate, and the first electrode (a transparent electrode:ITSO, 110 nm), a hole injecting layer (PStDPA, 30 nm), a hole transporting layer (α-NPD, 20 nm), an electron transporting layer and a light-emitting layer (Alq₃, 50 nm) and the second electrode (Al—Li, 50 nm) were stacked over the glass substrate.

Then, a manufacturing method of the light-emitting element is described. The first electrode was formed over the glass substrate first. In this example, the first electrode was formed of ITSO by a sputtering method. The shape of the first electrode was 2 mm×2 mm in the present invention.

Next, as a pretreatment for forming the PStDPA thin film over the first electrode, the surface of the substrate was washed with a porous resin (typically, one made of PVA (polyvinyl alcohol), nylon, or the like), heated at 200° C. for 1 hour, and subjected to a UV ozone treatment for 370 seconds.

After the pretreatment, a solution in which 150 mg of PStDPA was dissolved in 30 ml of toluene was filtered through a 5 μm filter, and the filtrate was applied to coat the first electrode by a spin-coating method. The application by a spin coating was conducted as follows: rotation at 500 rpm was conducted for 2 seconds and then, rotation at 600 rpm was conducted for 60 seconds. The glass substrate coated with the PStDPA was baked at 120° C. for 1 hour, and the PStDPA thin film was completed.

After forming the PStDPA thin film, α-NPD was formed as a hole transporting layer to have a thickness of 20 nm, Alq₃ was formed as the light-emitting layer also serving as the electron transporting layer to have a thickness of 50 nm, each of which was formed by a vacuum evaporation method using resistant heating. Further, after forming the Alq₃, Al—Li was deposited to have a thickness of 150 nm as the second electrode by a vacuum evaporation method to complete the light-emitting element of the present invention.

The current efficiency in light-emission of the thusly formed light-emitting element was measured. At this time, the measurement was conducted using a 2400 type Source Meter manufactured by Keithley Instruments Inc, by applying voltage while using the first electrode as an anode, and the second electrode as a cathode. The voltage was applied at 0.2 V intervals in the range of 0V to 20 V, and a current value and a luminance of each voltage value were measured. The luminance was measured using a color luminance meter BM-5A manufactured by Topcon Technohouse Corporation.

According to the measurement result, the maximum current efficiency ratio of the light-emitting element using PStDPA as hole injecting layer was about 3.7 cd/A.

FIG. 15 also shows a luminance property of a light-emitting element which was formed by using 4,4′-bis[N-(4-(N,N-di-m-tolylamino)phenyl)-N-phenylamino] biphenyl (DNTPD) for the hole injecting layer, as a comparative example.

The light-emitting element of this example as the comparative example, which was used for measuring a luminescence property was formed over a glass substrate, and a first electrode (a transparent electrode: ITSO, 110 nm), a hole injecting layer (DNTPD, 30 nm), a hole transporting layer (α-NPD, 20 nm), a light-emitting layer also serving as an electron transporting layer (Alq₃, 50 nm) and a second electrode (Al—Li, 50 nm) were stacked sequentially over the glass substrate. The only difference between the light-emitting element of Example 4 and the light-emitting element of the comparative example is the material for the hole transporting layer.

Then, a manufacturing method of the element for the comparative example is described. The first electrode was formed over the glass substrate first. In this example, the first electrode was formed of ITSO by a sputtering method. The shape of the first electrode was 2 mm×2 mm in the present invention.

Next, as a pretreatment for forming the DNTPD thin film over the first electrode, the surface of the substrate was washed with a porous resin (typically, one made of PVA (polyvinyl alcohol), nylon, or the like), heated at 200° C. for 1 hour, and subjected to a UV ozone treatment for 370 seconds.

After the pretreatment, DNTPD was formed as a hole injecting layer to have a thickness of 30 nm, α-NPD was formed as the hole transporting layer to have a thickness of 20 nm, Alq₃ was formed as the light-emitting layer also serving as the electron transporting layer to have a thickness of 50 nm, each of which was formed sequentially by a vacuum evaporation method using resistant heating. Further, after forming the Alq₃, Al—Li was deposited to have a thickness of 150 nm as the second electrode by a vacuum evaporation method to complete the light-emitting element as the comparative example of the present invention.

The thusly formed light-emitting element was measured in the same manner as conducted for the element of Example 4. The maximum current efficiency ratio of the light-emitting element as the comparative example was about 3.0 cd/A according to the measurement result.

According to the results of Example 4 and the comparative example, it is proved that the light-emitting element using PStDPA as the hole injecting layer provides a better current efficiency than the light-emitting element using DNTPD.

1. A hole injecting material comprising:
a polymer having a repeating unit represented by a following general formula (1): 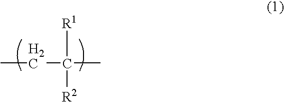
wherein R¹ represents hydrogen, an alkyl group, a cyano group, or an alkoxy group,
wherein R² represents an aryl group,
wherein the polymer has an ionization potential of 4.9 eV or more and 5.4 eV or less.

2. The hole injecting material according to claim 1, wherein the aryl group represented by R² in the formula has an electron donating substituent.

3. The hole injecting material according to claim 2, wherein the electron donating substituent has a substituent constant value σ of −2.1 or more and 0.15 or less in Hammett rule.

4. The hole injecting material according to claim 1, wherein the aryl group represented by R² in the formula is an aryl group having diarylamino.

5. A hole injecting material comprising:
a polymer having a repeating unit represented by a following general formula (1), 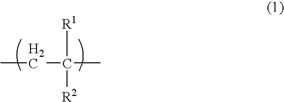
wherein R¹ represents hydrogen, an alkyl group, a cyano group, or an alkoxy group,
wherein R² represents a group represented by a following formula (2), and 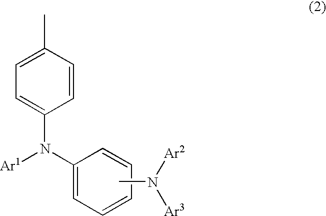
wherein each of Ar¹ to Ar³ represents an aryl group having carbon atoms 6 to 14.

6. A hole injecting material according to claim 5, wherein the polymer is a linear polymer.

7. A hole injecting material comprising:
a polymer having a repeating unit represented by a following general formula (3),

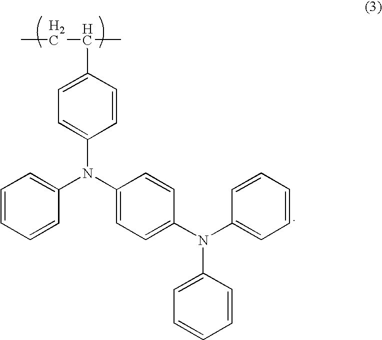

8. The hole injecting material according to any one of claims 1, 5 and 7, wherein the polymer is poly{4-[N-(4-diphenylaminophenyl)-N-phenyl]aminostyrene}.

9. The hole injecting material according to any one of claims 5 and 7, wherein the polymer has an ionization potential of 4.9 eV or more and 5.4 eV or less.

10. The hole injecting material according to any one of claims 1, 5 and 7, wherein the polymer has a number average molecular weight of 2000 or more and 500000 or less.

11. The hole injecting material according to any one of claims 1, 5 and 7, wherein the polymer has a number average molecular weight of 10000 or more and 100000 or less.

12. A light-emitting element using the hole injecting material according to claims 1, 5 and 7.

13. An organic compound represented by a following general formula (4), 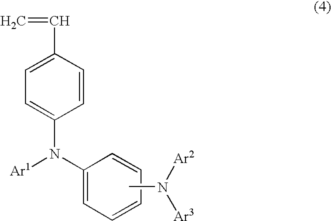
wherein each of Ar¹ to Ar³ represents an aryl group having carbon atoms 6 to 14.

14. A monomer comprising an organic compound represented by a following general formula (4), 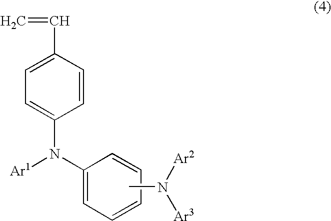
wherein each of Ar¹ to Ar³ represents an aryl group having carbon atoms 6 to 14.

15. A monomer mixture including at least two types of organic compounds represented by a following general formula (4), 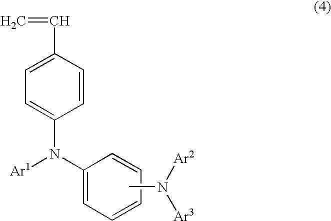
wherein each of Ar¹ to Ar³ represents an aryl group having carbon atoms 6 to 14.

16. An organic compound represented by a following general formula (5),

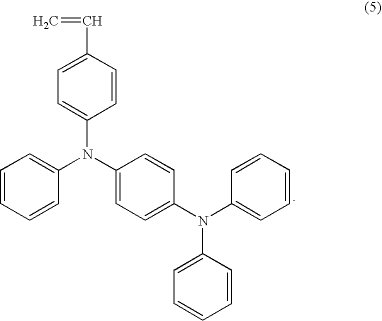

17. A monomer mixture comprising an organic compound represented by a following general formula (5),

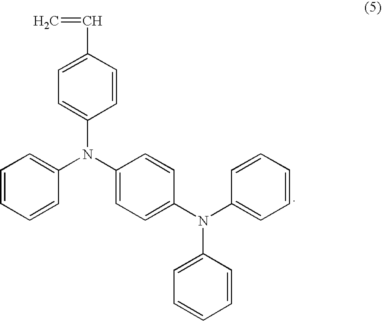

18. A monomer including an organic compound represented by a following general formula (5), [chemical structure]
